(19) United States
(12) Patent Application Publication
(10) Pub. No.: US 2023/0020342 A1
(43) Pub. Date: Jan. 19, 2023

(54) METHOD FOR PRODUCING POLYOL-BASED ESTERS, IN PARTICULAR POLYGLYCEROL ESTERS, FROM HYDROXY CARBOXYLIC ACIDS

(71) Applicant: IOI Oleo GmbH, Witten (DE)

(72) Inventors: Dirk LOCHMANN, Witten (DE); Sebastian REYER, Witten (DE); Michael STEHR, Witten (DE)

(21) Appl. No.: 17/618,619

(22) PCT Filed: Jun. 12, 2019
(86) PCT No.: PCT/EP2019/065271

§ 371 Date: Dec. 13, 2021

(51) Int. Cl. C07C 67/31; A23L 33/12

(52) U.S. Cl. CPC C07C 67/31; A23L 33/12

(57) ABSTRACT

The invention relates to a method for producing optionally functionalized, preferentially optionally fatty acid functionalized, polyol esters, especially polyglycerol esters, of 3-hydroxybutyric acid, as well as to the products thus obtained and their use.

### CROSS-REFERENCES TO RELATED APPLICATIONS

This application is a National Stage filing of International Application PCT/EP 2019/065271 filed Jun. 12, 2019, entitled “METHOD FOR PRODUCING POLYOL-BASED ESTERS, ESPECIALLY POLYGLYCEROL ESTERS, OF HYDROXYCARBOXYLIC ACIDS”. The subject application claims priority to PCT/EP 2019/065271 and incorporates all by reference herein, in its entirety.

### BACKGROUND OF THE INVENTION

The present invention relates to the field of keto bodies and related metabolism and the therapy of related diseases.

Especially, the present invention relates to a method for producing optionally functionalized, preferentially optionally fatty acid functionalized, preferably optionally C₅-C₃₄-fatty acid functionalized, especially optionally C₈-C₃₄-fatty acid functionalized, polyol esters, especially polyglycerol esters, of 3-hydroxybutyric acid as well as the reaction products thus obtainable or thus produced (i. e. optionally functionalized, preferentially optionally fatty acid functionalized, preferably optionally C₅-C₃₄-fatty acid functionalized, especially optionally C₅-C₃₄-fatty acid functionalized, polyol esters, especially polyglycerol esters, of 3-hydroxybutyric acid, preferably in enantiomerically enriched or enantiomerically pure form) and their functionalized derivatives as well as their use, especially in pharmaceutical compositions, such as drugs or medicaments, or in food and/or food products, as well as their further applications or uses.

Furthermore, the present invention relates to pharmaceutical compositions, especially drugs or medicaments, comprising the reaction products (i. e. optionally functionalized, preferentially optionally fatty acid functionalized, preferably optionally C₅-C₃₄-fatty acid functionalized, especially optionally C₈-C₃₄-fatty acid functionalized, polyol esters, especially polyglycerol esters, of 3-hydroxybutyric acid, preferably in enantiomerically enriched or enantiomerically pure form) obtainable or produced according to the inventive method, as well as their applications or uses.

Finally, the present invention relates to food and/or food products, especially food supplements, functional foods, novel foods, food additives, food supplements, dietary foods, power snacks, appetite suppressants and strength and/or endurance sports supplements, which comprise the reaction products (i. e. optionally functionalized, preferentially optionally fatty acid functionalized, preferably optionally C₅-C₃₄-fatty acid functionalized, especially optionally C₈-C₃₄-fatty acid functionalized, polyol esters, especially polyglycerol esters, of 3-hydroxybutyric acid, preferably in enantiomerically enriched or enantiomerically pure form) obtainable or produced according to the inventive method, as well as their applications or uses.

The term optionally functionalized, preferentially optionally fatty acid functionalized, preferably optionally C₅-C₃₄-fatty acid functionalized, especially optionally C₈-C₃₄-fatty acid functionalized, polyol esters, especially polyglycerol esters, of 3-hydroxybutyric acid, as used according to the invention, refers especially to compounds whose hydroxyl groups are optionally functionalized, especially optionally fatty acid functionalized, preferably optionally C₅-C₃₄-fatty acid functionalized, more preferably optionally C₈-C₃₄-fatty acid functionalized, (i. e. the hydrogen atom of the hydroxyl group(s) of the polyol and/or of the 3-hydroxybutyric acid is optionally replaced by an acyl group, i. e. a group —CH(OR)— with R=acyl, especially R═(C₅-C₃₄-alkyl)-C(O)—, preferentially R═(C₈-C₃₄-alkyl)-C(O)—, so that an ester group is present). Consequently, the reaction products according to the invention (i. e. optionally functionalized, preferentially optionally fatty acid functionalized, preferably optionally C₅-C₃₄-fatty acid functionalized, especially optionally C₈-C₃₄-fatty acid functionalized, polyol esters, especially polyglycerol esters, of 3-hydroxybutyric acid) may optionally undergo acylation on free hydroxyl groups of the polyol and/or the 3-hydroxybutyric acid or the 3-hydroxybutyrate (i. e. acylation in the 3-position of the 3-hydroxybutyrate radical in the ester concerned).

In the human energy metabolism, glucose is the short-term available energy carrier, which is metabolized into energy in the mitochondria by releasing water and carbon dioxide. The glycogen stores of the liver are already emptied during the sleep period during the night. However, especially the human central nervous system (CNS) and the heart require a permanent energy supply.

The physiological alternative to glucose, which is mainly available to the central nervous system, are the so-called keto bodies (synonymously also called ketone bodies or “ketone bodies” in English).

The term keto body is especially a collective term for three compounds, which are formed mainly in catabolic metabolic states (such as hunger, reduction diets or low-carbohydrate diets) and may lead to ketosis. The term keto bodies includes especially the three compounds acetoacetate (synonymously also referred to as acetacetate or 3-oxobutyrate) and acetone as well as 3-hydroxybutyric acid (hereinafter also synonymously referred to as beta-hydroxybutyric acid or BHB or 3-BHB) or its salt (i. e. 3-hydroxybutyrate or beta-hydroxybutyrate), wherein the latter is the most important of the three aforementioned compounds. 3-Hydroxybutyric acid or its salt occurs physiologically as the (R)-enantiomer, i. e. as (R)-3-hydroxybutyric acid (synonymously also called (3R)-3-hydroxybutyric acid to emphasize the center of chirality in the 3-position) or its salt.

These keto bodies are also provided physiologically in large amounts from lipids stored in the body by lipolysis during fasting or starvation and replace the energy source glucose almost completely.

The keto bodies are formed in the liver from acetyl coenzyme A (=acetyl-CoA), which originates from beta-oxidation; they represent a transportable form of the acetyl coenzyme A in the human body. However, in order to utilize the keto bodies, the brain and muscles must first adapt by expressing enzymes that are required to convert keto bodies back into acetyl coenzyme A. Especially in times of hunger, the keto bodies contribute a considerable amount to energy production. For example, after some time the brain is able to get by with only a third of the daily amount of glucose.

Physiologically, the keto bodies are synthesized from two molecules of activated acetic acid in the form of acetyl coenzyme A, the normal intermediate product of fatty acid degradation, which is extended using a further acetyl coenzyme A unit and the enzyme HMG-CoA-synthase to the intermediate product 3-hydroxy-3-methyl-glutaryl-CoA (HMG-CoA), wherein finally the HMG-CoA-lyase cleaves off the acetoacetate. These three steps take place exclusively in the mitochondria of the liver (lynen cycle), wherein 3-hydroxybutyrate is finally formed in the cytosol by the D-beta-hydroxybutyrate dehydrogenase. HMG-CoA is also an end product of the degradation of the amino acid leucine, while acetoacetate is formed during the degradation of the amino acids phenylalanine and tyrosine.

By spontaneous decarboxylation acetoacetate is formed from acetone; it can occasionally be perceived in the breath of diabetics and dieters. It cannot be further used by the body. However, the proportion of acetone in the keto bodies is small.

Acetoacetate is thus reductively converted into the physiologically relevant form of 3-hydroxybutyric acid or 3-hydroxybutyrate, but can also decompose into the physiologically unusable acetone with the release of carbon dioxide, which is detectable and olfactory perceptible in severe ketosis, a ketoacidosis (e. g. in diabetes mellitus type 1 patients without insulin substitution), in the urine and in the exhaled air.

3-Hydroxybutyric acid itself is currently used and marketed in the weight training sector as a sodium, magnesium or calcium salt.

However, 3-hydroxybutyric acid itself is not known or only in very small quantities to humans in evolutionary terms, since plants do not produce 3-hydroxybutyric acid and 3-hydroxybutyric acid in the animal organism only occurs in dead emaciated animals in ketosis, so that 3-hydroxybutyric acid causes nausea when administered orally. 3-Hydroxybutyric acid in the form of free acid and its salts also taste very bitter and can cause severe vomiting and nausea.

Moreover, patients, especially newborns, but also adults cannot permanently tolerate large amounts of salts of 3-hydroxybutyric acid, as these compounds can have a kidney-damaging effect.

In addition, the plasma half-life of 3-hydroxybutyric acid and its salts is so short that even if several grams are taken, the ketosis lasts only for about three to four hours, i. e. patients cannot benefit continuously from a therapy with 3-hydroxybutyric acid or its salts, especially at night. In case of metabolic diseases this can lead to life-threatening situations.

Therefore, in the case of the therapy of such metabolic diseases, so-called medium-chain triglycerides, so-called MCTs, are currently used for ketogenic therapy, i. e. the metabolic conversion of caproic, caprylic and capric acid (i. e. of saturated linear C₆-, C₈- and C₁₀-fatty acids) from the corresponding triglycerides is intended.

Basically, however, from a pharmaceutical and clinical point of view, 3-hydroxybutyric acid is a more effective pharmaceutical-pharmacological target molecule, which, according to the prior art, could in principle be used for the therapy of a large number of diseases, but cannot be used due to its lack of physiological compatibility (e. g. in diseases in connection with a malfunction of the energy metabolism, especially keto-body metabolism, or neurodegenerative diseases such as dementia, Alzheimer's disease, Parkinson's disease, etc., lipometabolic diseases etc.).

The following table illustrates purely exemplary, but by no means limiting, potential therapy options or possible indications for the active ingredient 3-hydroxybutyric acid.

| Indication | Therapeutic effect |
|---|---|
| Traumatic | Under BHB the apoptosis and |
| brain injury | necrosis rate of nerve cells decreases. |
| Stroke | Under BHB the apoptosis and |
| | necrosis rate of nerve cells decreases. |
| Refeeding | In case of anorexia, discontinuation |
| syndrome | of enteral or parenteral nutrition |
| | and after long periods of hunger, |
| | the consumption of starch or glucose |
| | can lead to death (see also WHO |
| | scheme peanut paste). BHB can be used |
| | here as a therapeutic agent to achieve |
| | normal food intake more quickly. |
| Appetite | BHB suppresses the feeling of hunger |
| suppressant | in the central nervous system (CNS). |
| Epilepsy | Conventional ketogenic diet to |
| | significantly reduce the frequency of |
| | seizures has extremely poor |
| | patient tolerance. BHB offers an |
| | immediately effective alternative here. |
| Alzheimer's | Under BHB patients show better |
| disease, | cognitive performance. BHB is also |
| dementia | effective in the prevention of |
| | neurodegenerative diseases. |
| Disorders of | Compensation of a nutrient |
| fatty acid | deficiency in case of defect in |
| oxidation | energy metabolism. |
| (e. g. electron | |
| transfer protein | |
| defect) | |

Therefore, it is desirable from a pharmaceutical and clinical point of view to be able to find effective precursors or metabolites which physiologically allow direct or indirect access to 3-hydroxybutyric acid or its salts, especially in the physiological metabolism of the human or animal body.

Consequently, the prior art has not lacked attempts to find physiologically suitable precursors or metabolites for 3-hydroxybutyric acid or its salts. So far, however, no efficient compounds have been found in the prior art. Also, access to such compounds is not or not readily possible according to the prior art.

### BRIEF SUMMARY OF THE INVENTION

The problem underlying the present invention is thus the provision of an efficient method for producing physiologically suitable or physiologically compatible precursors and/or metabolites of 3-hydroxybutyric acid (i. e. beta-hydroxybutyric acid or BHB or 3-BHB) or their salts.

Such method should especially make the respective BHB precursors and/or BHB metabolites accessible in an efficient way, especially in larger quantities and without significant amounts of toxic by-products.

In a completely surprising way, the applicant has now discovered that optionally functionalized, preferentially optionally fatty acid functionalized, preferably optionally C₅-C₃₄-fatty acid functionalized, especially optionally C₅-C₃₄-fatty acid functionalized, polyol esters, especially polyglycerol esters, of 3-hydroxybutyric acid (beta-hydroxybutyric acid, BHB or 3-BHB) represent an efficient and physiologically effective or physiologically compatible precursor and/or metabolite for the keto body 3-hydroxybutyric acid or its salts and has in this context been able to find or develop an efficient method for producing these compounds, which allows direct and effective, especially economic as well as industrially feasible access to these compounds.

To solve the problem described above, the present invention therefore proposes—according to a first aspect of the present invention—a method for producing optionally functionalized, preferentially optionally fatty acid functionalized, preferably optionally C₅-C₃₄-fatty acid functionalized, especially optionally C₈-C₃₄-fatty acid functionalized, polyol esters, especially polyglycerol esters, of 3-hydroxybutyric acid (beta-hydroxybutyric acid, BHB or 3-BHB) is provided; further, especially special and/or advantageous embodiments of the inventive method are similarly described.

Furthermore, the present invention relates—according to a second aspect of the present invention—to a reaction product obtainable according to the inventive method or an optionally functionalized, preferentially optionally fatty acid functionalized, preferably optionally C₅-C₃₄-fatty acid functionalized, especially optionally C₈-C₃₄-fatty acid functionalized, polyol ester, especially polyglycerol ester, of 3-hydroxybutyric acid (beta-hydroxybutyric acid, BHB or 3-BHB) or a mixture of at least two, preferentially at least three, different optionally functionalized, preferentially optionally fatty acid functionalized, preferably optionally C₅-C₃₄-fatty acid functionalized, especially optionally C₈-C₃₄-fatty acid functionalized, polyol esters, especially polyglycerol esters, of 3-hydroxybutyric acid (beta-hydroxybutyric acid, BHB or 3-BHB) obtainable in this regard; further, especially special and/or advantageous embodiments of this aspect of the invention are provided.

Likewise, the present invention—according to a third aspect of the present invention—relates to a pharmaceutical composition, especially a drug or medicament; further, especially special and/or advantageous embodiments of this aspect of the invention are provided.

Furthermore, the present invention—according to a fourth aspect of the present invention—relates to an inventive reaction product or an inventive polyol ester, especially polyglycerol ester, of 3-hydroxybutyric acid (beta-hydroxybutyric acid, BHB or 3-BHB) or an inventive mixture of at least two, preferentially at least three polyol esters, especially polyglycerol esters, of 3-hydroxybutyric acid (beta-hydroxybutyric acid, BHB or 3-BHB) for the prophylactic and/or therapeutic treatment or for use in the prophylactic and/or therapeutic treatment of diseases of the human or animal body.

Furthermore, the present invention—according to a fifth aspect of the present invention—relates to the use of an inventive reaction product or an inventive polyol ester, especially polyglycerol ester, of 3-hydroxybutyric acid (beta-hydroxybutyric acid, BHB or 3-BHB) or of an inventive mixture of at least two, preferentially at least three polyol esters, especially polyglycerol esters, of 3-hydroxybutyric acid (beta-hydroxybutyric acid, BHB or 3-BHB) for the prophylactic and/or therapeutic treatment or for producing a medicament for the prophylactic and/or therapeutic treatment of diseases of the human or animal body.

Furthermore, the present invention—according to a sixth aspect of the present invention—relates to the use of an inventive reaction product or an inventive polyol ester, especially polyglycerol ester, of 3-hydroxybutyric acid (beta-hydroxybutyric acid, BHB or 3-BHB) or an inventive mixture of at least two, preferentially at least three polyol esters, especially polyglycerol esters, of 3-hydroxybutyric acid (beta-hydroxybutyric acid, BHB or 3-BHB) for the prophylactic and/or therapeutic treatment or for producing a medicament for the prophylactic and/or therapeutic treatment of or for the application for catabolic metabolic states, such as hunger, diets or low-carbohydrate nutrition are provided.

Furthermore, the present invention—according to a seventh aspect of the present invention—relates to a food and/or food product; further, especially special and/or advantageous embodiments of the food and/or food product according to the invention are provided.

Finally, the present invention—according to an eighth aspect of the present invention—relates to the use of an inventive reaction product or an inventive polyol ester, especially polyglycerol ester, of 3-hydroxybutyric acid (beta-hydroxybutyric acid, BHB or 3-BHB) or of an inventive mixture of at least two, preferentially at least three polyol esters, especially polyglycerol esters, of 3-hydroxybutyric acid (beta-hydroxybutyric acid, BHB or 3-BHB) in a food and/or a food product; further, especially special and/or advantageous embodiments of the use according to the invention are provided.

It goes without saying that following features, embodiments, advantages and the like, which are subsequently listed below only with regard to one aspect of the invention for the purpose of avoiding repetition, naturally also apply accordingly to the other aspects of the invention, without this requiring a separate mention.

Furthermore, it goes without saying that individual aspects and embodiments of the present invention are also considered disclosed in any combination with other aspects and embodiments of the present invention and, especially, any combination of features and embodiments, as it results from back references of all patent claims, is also considered extensively disclosed with regard to all resulting combination possibilities.

With respect to all relative or percentage weight-based data provided below, especially relative quantity or weight data, it should further be noted that within the scope of the present invention these are to be selected by the person skilled in the art such that they always add up to 100% or 100% by weight, respectively, including all components or ingredients, especially as defined below; however, this is self-evident for the person skilled in the art.

In addition, the skilled person may, if necessary, deviate from the following range specifications without leaving the scope of the present invention.

Additionally, it applies that all values or parameters or the like specified in the following can be determined or identified in principle with standardized or explicitly specified determination methods or otherwise with the determination or measurement methods that are otherwise familiar to a person skilled in the art.

Having stated this, the present invention will be described in more detail hereinafter:

### DETAILED DESCRIPTION OF THE INVENTION

The subject-matter of the present invention—according to a first aspect of the present invention—is thus a method for producing a polyol ester, especially polyglycerol ester, of 3-hydroxybutyric acid (beta-hydroxybutyric acid, BHB or 3-BHB), which polyol ester is optionally functionalized, preferentially optionally fatty acid functionalized, preferably optionally C₅-C₃₄-fatty acid functionalized, especially optionally C₅-C₃₄-fatty acid functionalized,

wherein an optionally functionalized, preferentially optionally fatty acid functionalized, preferably optionally C₅-C₃₄-fatty acid functionalized, especially optionally C₈-C₃₄-fatty acid functionalized, 3-oxobutyric acid polyol ester (I), especially 3-oxobutyric acid polyglycerol ester, which comprises at least one 3-oxobutyrate radical (3-oxobutyric acid radical) of the general formula (I′)

CH₃—C(O)—CH₂—C(O)O— (I′)

is subjected, by means of at least one reducing agent, to a preferably selective reduction of the at least one 3-oxobutyrate radical of the general formula (I′) positioned at the keto group —C(O)— of the acetyl function CH₃— C(O)— so as to result a 3-hydroxybutyrate radical (3-hydroxybutyric acid radical) of the general formula (II′)

CH₃—CH(OH)—CH₂—C(O)O— (II′)

so that, as a reaction product, an optionally functionalized, preferentially optionally fatty acid functionalized, preferably optionally C₅-C₃₄-fatty acid functionalized, especially optionally C₅-C₃₄-fatty acid functionalized, 3-hydroxybutyric acid polyol ester (II), especially 3-hydroxybutyric acid polyglycerol ester, is obtained.

As stated above, the applicant has, quite surprisingly, discovered that the optionally functionalized, preferentially optionally fatty acid functionalized, preferably optionally C₅-C₃₄-fatty acid functionalized, especially optionally C₅-C₃₄-fatty acid functionalized, polyol esters, especially polyglycerol esters, of 3-hydroxybutyric acid (beta-hydroxybutyric acid, BHB or 3-BHB) thus produced are efficient, since physiologically compatible precursors and/or metabolites of 3-hydroxybutyric acid or their salts, which can also be used in larger quantities in pharmaceutical or clinical applications because they are physiologically compatible.

The above-mentioned optionally functionalized polyol esters, especially polyglycerol esters, of 3-hydroxybutyric acid, which are accessible in an efficient manner through the production method according to the invention, represent a physiologically and pharmacologically relevant alternative to free 3-hydroxybutyric acid or its salts.

The production of optionally functionalized polyol esters, especially polyglycerol esters, of 3-hydroxybutyric acid by means of conventional organic synthesis is complex and costly, since 3-hydroxybutyric acid has an increased tendency to polymerize and to undergo other undesirable side reactions (e. g. dehydration, decomposition, etc.). Within the scope of the present invention, it was possible to provide an efficiently working production method with which optionally functionalized polyol esters, especially polyglycerol esters, of 3-hydroxybutyric acid can be produced without undesired side reactions.

The inventive method thus makes it possible to provide non-toxic optionally functionalized polyol esters, especially polyglycerol esters, of 3-hydroxybutyric acid from physiologically harmless components or reactants (starting compounds). The resulting optionally functionalized polyol esters, especially polyglycerol esters, of 3-hydroxybutyric acid can be broken down physiologically, especially in the stomach and/or bowl, and release or generate the target molecule “3-hydroxybutyric acid” or its salts as active ingredient or active component.

In addition, the aforementioned optionally functionalized polyol esters, especially polyglycerol esters, of 3-hydroxybutyric acid also comprise an acceptable taste to ensure compatibility even when administered orally in larger quantities over a longer period of time (e. g. administration of 50 g daily dose or more).

Similarly, the production method according to the invention makes it possible to provide the optionally functionalized polyol esters, especially polyglycerol esters, of 3-hydroxybutyric acid free from toxic impurities.

In addition, the method can also be carried out enantioselectively, especially by means of chiral catalysis. For example, according to the invention, the production method allows the biologically relevant form, i. e. the (R)-enantiomer, to be enriched or to be solely produced as not to burden the renal system of patients when administered orally (i. e. elimination via the kidneys). In principle, however, it is also possible, and under certain conditions may be useful, to enrich the (S)-enantiomer or to solely produce it.

In addition, the production method according to the invention, including optional further processing or purification steps, can be operated economically and can also be implemented on a large scale.

Especially, the inventive production method uses easily available starting compounds and furthermore allows a relatively simple process management even in case of large-scale implementation.

Nevertheless, excellent yields are achieved in accordance with the invention, wherein the formation of by-products is minimized (no significant amounts of by-products) or avoided.

In contrast to conventional prior art production methods, the production method according to the invention does not use complex starting materials and uses only a single step.

In contrast to conventional prior art production methods, the starting materials are also physiologically compatible and even pharmaceutically effective, i. e. any unreacted starting material can remain in the end product, meaning that no or hardly any purification steps are necessary (even if removal is easily possible, if desired). Especially, the method according to the invention is usually carried out in the absence of solvents and/or without any solvent (i. e. as a reaction in mass or as a reaction in substance or as a so-called bulk reaction); consequently, the reaction products obtained are not contaminated with solvent and no solvent has to be removed and disposed of or recycled in a costly and energy-intensive manner after the method or reaction has been carried out. Furthermore, no toxic by-products are formed.

In addition, the production method according to the invention uses commercially available, non-toxic and pharmacologically compatible reducing agents.

According to a particular embodiment, the production method according to the invention typically results in a mixture of different optionally functionalized polyol esters, especially polyglycerol esters, of 3-hydroxybutyric acid, i. e. in a mixture of at least two, especially at least three different optionally functionalized polyol esters, especially polyglycerol esters, of 3-hydroxybutyric acid. The resulting raw reaction product or raw mixture can, if required, be purified by known methods, especially by removing any remaining starting compounds and/or any by-products present, and furthermore—if desired—can be separated by known methods, especially by distillation and/or chromatography (e. g. fractionation into the individual optionally functionalized polyol esters, i. e. mono-, di-, tri- etc. optionally functionalized polyol esters of 3-hydroxybutyric acid, or else fractionation into fractions with enriched and depleted portions of individual optionally functionalized polyol esters etc.).

However, if necessary, the production method according to the invention can also be used to produce the pure or individual optionally functionalized polyol esters, especially polyglycerol esters, of 3-hydroxybutyric acid as pure or individual substances (e. g. depending on the reactants used).

Furthermore, the production method according to the invention allows a targeted partial reduction (hydrolysis) by controlling the reaction, especially the amount of reducing agent; in other words, if necessary, only a certain, especially defined proportion of the 3-oxobutyrate radicals or the keto groups —C(O)— contained therein can be converted to a 3-hydroxybutyrate radical or the corresponding hydroxyl function—CH(OH)—. Hereby it is possible to provide a reaction product which exhibits a further retard effect. Due to the presence of both 3-oxobutyrate radicals and 3-hydroxybutyrate radicals in a corresponding product mixture, there is a different rate of availability or release of the active ingredient 3-hydroxybutyric acid.

Within the production method according to the invention, only the keto group—C(O)— of the acetyl function CH—C(O)—is selectively reduced to a hydroxyl group —CH(OH)—, i. e. no side reactions, especially no rearrangements, cleavages, additions, etc., are taking place.

In contrast to conventional production methods of the prior art, the production method according to the invention does not result in a dimerization of the 3-hydroxybutyric acid, as occurs, for example, in the direct reaction of 3-hydroxybutyric acid with a polyol ester.

Overall, the method according to the invention is thus both simple and economical or economic and thus can also be operated on a large scale.

In other words, the present invention thus relates to a method for producing an optionally functionalized, preferentially optionally fatty acid functionalized, preferably optionally C₅-C₃₄-fatty acid functionalized, especially optionally C₈-C₃₄-fatty acid functionalized, polyol ester, especially polyglycerol ester, of 3-hydroxybutyric acid (beta-hydroxybutyric acid, BHB or 3-BHB),

wherein an optionally functionalized, preferentially optionally fatty acid functionalized, preferably optionally C₅-C₃₄-fatty acid functionalized, especially optionally C₅-C₃₄-fatty acid functionalized, 3-oxobutyric acid polyol ester (I), especially 3-oxobutyric acid polyglycerol ester, which comprises at least one 3-oxobutyrate radical (3-oxobutyric acid radical) of the general formula (I′)

CH₃—C(O)—CH₂—C(O)O— (I′)

is subjected, by means of at least one reducing agent, to a preferably selective reduction of the keto group —C(O)— of the acetyl function CH₃— C(O)— of the at least one 3-oxobutyrate radical of the general formula (I′) so as to result a hydroxyl function—CH(OH)—,
so that, as a reaction product, an optionally functionalized, preferentially optionally fatty acid functionalized, preferably optionally C₅-C₃₄-fatty acid functionalized, especially optionally C₅-C₃₄-fatty acid functionalized, 3-hydroxybutyric acid polyol ester (II), especially 3-hydroxybutyric acid polyglycerol ester, is obtained

In the context of the invention, the term selective reduction is to be understood especially only as the reduction of the keto group —C(O)— of the acetyl function CH₃— C(O)— of the 3-oxobutyrate radical of the general formula (I′), as defined hereinabove, to a hydroxyl group —CH(OH)—of the 3-hydroxybutyrate radical of the general formula (II′), as defined hereinabove. Especially, no side reactions, especially no rearrangements, cleavages, additions, etc., take place during this selective reduction. Furthermore, no other functional group (i. e. no functional group other than the aforementioned keto group) is reduced.

Also, in the course of chiral catalysis, as will be explained in detail below, the reaction product can be obtained in an enantiomerically enriched form or preferably even in an enantiomerically pure form (e. g. at least 95%, especially at least 99% enantiomeric purity) (e. g. as (R)-enantiomer according to below explained general formula (II′)).

The reducing agent which can be used in the method according to the invention can be varied within a wide range. According to the invention, it is preferred if the reducing agent is selected from the group of hydrogen, hydrides, especially inorganic hydrides, and alcohols, especially C₁-C₃₄-alcohols, as well as mixtures thereof, preferentially selected from the group of hydrogen, alkali metal or alkaline earth metal borohydride, alkali metal or alkaline earth metal hydride, alkali metal or alkaline earth metal aluminum hydride, methanol, ethanol, propanol and isopropanol as well as mixtures thereof, preferably from the group of hydrogen, alkali metal or alkaline earth metal borohydride and isopropanol.

In the context of the present invention, the reduction may be carried out autocatalytically or in the presence of a catalyst.

Especially, in the method according to the invention, in case of carrying out the reduction in the presence of a catalyst, the catalyst is recycled after the reduction has been carried out

As previously stated, according to a particular embodiment of the production method of the invention, the reduction can be carried out autocatalytically.

In the case wherein the reduction is carried out autocatalytically, the reducing agent may especially be a hydride, preferentially an inorganic hydride, preferably alkali metal or alkaline earth metal borohydride, alkali metal or alkaline earth metal hydride and/or alkali metal or alkaline earth metal aluminum hydride, more preferably alkali metal or alkaline earth metal borohydride.

If the reduction of the inventive production method is carried out with a hydride, it is preferred if the reduction is carried out at temperatures in the range of from 2° C. to 30° C., especially in the range of from 3° C. to 25° C., preferentially in the range of from 3° C. to 20° C., preferably in the range of from 3° C. to 15° C., more preferably in the range of from 3° C. to 12° C.

When, according to a particular embodiment of the present invention, the reduction is carried out with a hydride, the applied pressure range can vary within a wide range. Especially, if the reduction is carried out with a hydride, the reduction can be carried out at a pressure in the range of from 0.0001 bar to 10 bar, especially in the range of from 0.001 bar to 5 bar, preferentially in the range of from 0.01 bar to 2 bar, more preferably in the range of from 0.05 bar to 1 bar, even more preferably at about 1 bar.

In case of using a hydride as a reducing agent, the amount of hydride used may vary within wide amounts. Especially, the hydride can be used in amounts, based on the amount of the optionally functionalized, preferentially optionally fatty acid functionalized, preferably optionally C₅-C₃₄-fatty acid functionalized, especially optionally C₈-C₃₄-fatty acid functionalized, 3-oxobutyric acid polyol ester (I), especially 3-oxobutyric acid polyglycerol ester, in the range of from 0.001% by weight to 20% by weight, especially in the range of from 0.01% by weight to 15% by weight, preferentially in the range of from 0.1% by weight to 15% by weight, preferably in the range of from 0.5% by weight to 10% by weight. Nevertheless, it may be necessary to deviate from the above-mentioned amounts in individual cases or for specific applications without leaving the scope of the present invention

According to an alternative embodiment of the present invention, the reduction may be carried out in the presence of a catalyst.

As shown hereinabove, if a catalyst is used, it is preferred to recycle it after the reduction has been carried out.

If the reduction in the inventive production method is carried in the presence of a catalyst, it is preferred if the catalyst is selected from the group of enzymes and/or metals or metal compounds, especially noble metals and transition metal compounds.

In this context, it is particularly preferred if the catalyst is selected from the group of dehydrogenases, especially alcohol dehydrogenases, and metals and/or metal compounds based on palladium, platinum, rhodium, iridium, ruthenium and nickel, preferentially from the group of dehydrogenases, especially alcohol dehydrogenases, and metals and/or metal compounds based on platinum, palladium, nickel and rhodium.

Dehydrogenases are enzymes that oxidize their substrate by splitting off hydrogen anions (i. e. H⁻). Consequently, dehydrogenases are not to be confused with dehydratases, which split off water. Dehydrogenases belong to group I (oxidoreductases) of the EC-classification of enzyme groups. The electrons as well as the split-off hydrogen are transferred to co-factors, such as NAD⁺ or FAD. Depending on the substrate, different dehydrogenases can be distinguished; for example, the enzyme that converts ethanol to acetaldehyde (ethanal) in the liver during alcohol degradation is an alcohol dehydrogenase (EC 1.1.1.1). Alcohol dehydrogenases (ADH) are enzymes that catalyze both the reaction of alcohols to the corresponding aldehydes or ketones and the reverse reaction (aldehyde or ketone to alcohol). This reaction is a redox reaction.

Inventively particularly suitable catalysts based on metals and/or metal compounds are the Wilkinson catalyst, which is a homogeneous catalyst with the empirical formula C₅₄H₄₅ClP₃Rh. This is a rhodium complex which finds application in hydrogenation, hydroformylation, hydrosilylation and for isomerization. Another catalyst particularly suitable according to the invention is Raney nickel, which is a solid catalyst consisting of fine grains of a nickel/aluminum alloy.

As mentioned hereinabove, according to a particular embodiment of the production method of the invention, the reduction may be carried out in the presence of an enzyme as a catalyst.

Preferentially, the enzyme may be a dehydrogenase, especially an alcohol dehydrogenase.

As stated hereinabove in connection with the use of a catalyst in general, in case of using an enzyme as a catalyst, it is preferred to recycle the enzyme after the reduction has been carried out.

If the reduction of the inventive production method is carried out in the presence of an enzyme as a catalyst, it is preferred if the reduction is carried out at temperatures in the range of from 5° C. to 80° C., especially in the range of from 10° C. to 65° C., preferentially in the range of from 10° C. to 50° C., preferably in the range of from 15° C. to 40° C., more preferably in the range of from 15° C. to 30° C.

In the context of this particular embodiment of the present invention, wherein the reduction is carried out in the presence of an enzyme as a catalyst, it is preferred if the reduction is carried out in the presence of an enzyme at a pressure in the range of from 0.0001 bar to 10 bar, especially in the range of from 0.001 bar to 5 bar, preferentially in the range of from 0.01 bar to 2 bar, more preferably in the range of from 0.05 bar to 1 bar, even more preferably at about 1 bar.

Nevertheless, it may be necessary to deviate from the above-mentioned temperature and pressure ranges in individual cases or for specific applications without leaving the scope of the present invention.

In the case of using an enzyme as a catalyst, the amount of enzyme used can vary within a wide range. Especially, the enzyme can be used in amounts, based on the total amount of starting compounds, in the range of from 0.001% by weight to 20% by weight, especially in the range of from 0.01% by weight to 15% by weight, preferentially in the range of from 0.1% by weight to 15% by weight, preferably in the range of from 0.5% by weight to 10% by weight. Nevertheless, it may be necessary to deviate from the above-mentioned amounts in individual cases or for specific applications without leaving the scope of the present invention. In this context, starting compounds are to be understood as the optionally functionalized, preferentially optionally fatty acid functionalized, preferably optionally C₅-C₃₄-fatty acid functionalized, especially optionally C₈-C₃₄-fatty acid functionalized, 3-oxobutyric acid polyol ester (I) and the reducing agent.

According to an alternative embodiment of the inventive method, the reduction may be carried out in the presence of a metal and/or a metal compound as a catalyst.

In this context, it is preferred if the metal and/or metal compound is selected from noble metals and transition metal compounds.

As stated hereinabove in connection with the use of a catalyst in general, in case of using a metal and/or metal compound as a catalyst, it is preferred to recycle the metal and/or metal compound after the reduction has been carried out.

According to this particular embodiment, the catalyst may be selected from the group of metals and/or metal compounds based on palladium, platinum, rhodium, iridium, ruthenium and nickel, preferentially from the group of metals and/or metal compounds based on platinum, palladium, nickel and rhodium.

If the reduction of the inventive method is carried out in the presence of a metal as a catalyst, the applied temperature range may vary within a wide range. Especially, the reduction in the presence of a metal as a catalyst can be carried out at temperatures in the range of from 10° C. to 140° C., especially in the range of from 15° C. to 135° C., preferentially in the range of from 20° C. to 130° C., preferably in the range of from 25° C. to 125° C., more preferably in the range of from 35° C. to 120° C., even more preferably in the range of from 40° C. to 110° C.

When, according to this particular embodiment of the present invention, the reduction is carried out in the presence of a metal as a catalyst, the applied pressure range can also vary within a wide range. Especially, if the reduction is carried out in the presence of a metal as a catalyst, the reduction may be carried out at a pressure in the range of from 2 bar to 80 bar, especially in the range of from 5 bar to 70 bar, preferentially in the range of from 10 bar to 60 bar, more preferably in the range of from 15 bar to 55 bar, even more preferably in the range of from 20 bar 50 bar.

In case of using a metal as a catalyst, the amount of the metal used can also vary within a wide range. Especially, the metal can be used in amounts, based on the total amount of starting compounds, in the range of from 0.001% by weight to 20% by weight, especially in the range of from 0.01% by weight to 15% by weight, preferentially in the range of from 0.1% by weight to 15% by weight, preferably in the range of from 0.5% by weight to 10% by weight.

Nevertheless, it may be necessary to deviate from the above-mentioned amounts in individual cases or for specific applications without leaving the scope of the present invention.

According to one embodiment of the present invention, the reduction of the optionally functionalized, preferentially optionally fatty acid functionalized, preferably optionally C₅-C₃₄-fatty acid functionalized, especially optionally C₈-C₃₄-fatty acid functionalized, 3-oxobutyric acid polyol ester (I), especially polyglycerol ester, can be carried out with hydrogen as a reducing agent in the presence of a metal and/or a metal compound based on palladium, platinum, rhodium, iridium, ruthenium and nickel as a catalyst.

According to an alternative embodiment of the present invention, the reduction of the optionally functionalized, preferentially optionally fatty acid functionalized, preferably optionally C₅-C₃₄-fatty acid functionalized, especially optionally C₈-C₃₄-fatty acid functionalized, 3-oxobutyric acid polyol ester (I), especially polyglycerol ester, can be carried out with a C₁-C₃₄-alcohol, especially isopropanol, as a reducing agent in the presence of dehydrogenase, especially alcohol dehydrogenase, as a catalyst.

According to another alternative embodiment of the present invention, the reduction of the optionally functionalized, preferentially optionally fatty acid functionalized, preferably optionally C₅-C₃₄-fatty acid functionalized, especially optionally C₈-C₃₄-fatty acid functionalized, 3-oxobutyric acid polyol ester (I), especially polyglycerol ester, can be carried out autocatalytically in the presence of alkali or alkaline earth metal borohydride as a reducing agent.

In the context of the present invention, the reduction of the keto group —C(O)— of the acetyl function CH₃— C(O)—in the 3-oxobutyrate radical(s) of the general formula (I′), as defined hereinabove, to a hydroxyl group may be carried out by means of chiral and/or enantioselective reaction control.

In this context, it is particularly preferred if the reduction is carried out in the presence of a chiral and/or enantioselective catalyst. Enantioselective or chiral catalysts can be, for example, the previously listed Wilkinson catalyst or a dehydrogenase, especially an alcohol dehydrogenase, such as the commercially available Chiralidon® R.

In other words, in this context, the reduction can especially be enantioselective.

Especially, in the method according to the invention, the optionally functionalized, preferentially optionally fatty acid functionalized, preferably optionally C₅-C₃₄-fatty acid functionalized, especially optionally C₅-C₃₄-fatty acid functionalized, 3-hydroxybutyric acid polyol ester (II), especially 3-hydroxybutyric acid polyglycerol ester, is formed enantiomerically enriched, especially enantiomerically pure, preferentially in the form of the (R)-configured enantiomer, each based on the chirality center (asymmetric carbon atom) of the 3-hydroxybutyrate radical of the general formula (II′) CH₃—C*H(OH)—CH₂— C(O)O—, which is generated by means of reduction and is subsequently identified by the symbol “*”.

In this context, it is particularly preferred if the (R)-enantiomer of the optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester (II), especially 3-hydroxybutyric acid polyglycerol ester, is formed, based on the chirality center (asymmetric carbon atom) of the 3-hydroxybutyrate radical of the general formula (II′) CH₃—C*H(OH)—CH₂— C(O)O—, which is generated by means of reduction and is subsequently identified by the symbol “*”.

In stereochemistry, chirality describes a spatial arrangement of atoms within a molecule in which simple symmetry operation, such as mirroring on a symmetry plane, does not result in self-imaging. A chirality center (synonymously called a stereocenter) is a point (especially an atom) in a molecule with a set of substituents in such a spatial arrangement that it cannot be made to coincide with the mirror image arrangement. Molecules that are mirror images of each other are called enantiomers and their respective chemical compounds are called chiral. In the present invention, the chirality center of the 3-hydroxybutyrate radical of the general formula (II′) is located at the C-atom in 3-position, which has a hydroxyl group as a substituent.

In the context of the present invention, enantiomerically enriched means the presence of at least 90%, especially at least 95%, preferentially at least 96%, preferably at least 97%, more preferably at least 98%, even more preferably at least 99%, of an enantiomer in the reaction product. Furthermore, in the context of the present invention, enantiomerically pure is to be understood as the presence of essentially 100% of an enantiomer in the reaction product.

According to the invention, however, according to an alternative embodiment or procedure, the reduction of the keto group —C(O)— of the acetyl function CH₃— C(O)—in the 3-oxobutyrate radical(s) of the general formula (I′), as defined hereinabove, to a hydroxyl group can be carried out by means of non-chiral and/or non-enantioselective reaction control.

In this context, it is advantageous if the reduction is carried out in the presence of a non-chiral and/or non-enantioselective catalyst. Such a catalyst is, for example, the previously mentioned Raney nickel or sodium borohydride (NaBH₄).

According to this alternative embodiment, especially a racemate (racemic mixture) of the optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester (II), especially 3-hydroxybutyric acid polyglycerol ester, is formed, each based on the chirality center (asymmetric carbon atom) of the 3-hydroxybutyrate radical of the above general formula (II′), as defined hereinabove, which is generated by means of reduction.

According to this embodiment, a racemic optionally functionalized, preferentially optionally fatty acid functionalized, preferably optionally C₅-C₃₄-fatty acid functionalized, especially optionally C₈-C₃₄-fatty acid functionalized, (R)/(S)-3-hydroxybutyric acid polyol ester (II), especially (R)/(S)-3-hydroxybutyric acid polyglycerol ester, is formed, each based on the chirality center (asymmetric carbon atom) of the 3-hydroxybutyrate radical of the above general formula (II′), as defined hereinabove, which is generated by means of reduction.

According to a particular embodiment of the present invention, the reduction may be carried out as a complete reduction of all keto groups —C(O)— of the acetyl function CH₃— C(O)— of the at least one 3-oxobutyrate radical of the general formula (I′), as defined hereinabove, to a 3-hydroxybutyrate radical of the general formula (II′), as defined hereinabove. Thus, according to this embodiment, the reduction may be carried out such that all keto groups —C(O)— of the acetyl function CH₃— C(O)— of the at least one 3-oxobutyrate radical of the general formula (I′), as defined hereinabove, are converted to a 3-hydroxybutyrate radical of the general formula (II′), as defined hereinabove.

In this context, it is preferred if the reduction is carried out such that the optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester (II), especially 3-hydroxybutyric acid polyglycerol ester, obtainable as a reaction product does not comprise any acetyl function CH₃— C(O)—.

In this context, it is preferred according to the invention if at least stoichiometric amounts of reducing agent, based on the keto groups to be reduced of the 3-oxobutyrate radical of the general formula (I′), are used.

Taking into account process economy and optimization of the process sequence, especially with regard to minimization of by-products, it is advantageous if the reducing agent is used in molar amounts, based on to the keto groups —C(O)— of the acetyl function CH₃— C(O)— of the at least one 3-oxobutyrate radical of the general formula (I′), in a range of from equimolar amount to a molar excess of 200 mol-%, especially in a range of from equimolar amount to a molar excess of 150 mol-%, preferentially in a range of from equimolar amount to a molar excess of 100 mol-%.

Likewise, taking into account process economy and optimization of the process sequence, especially with regard to minimization of by-products, it is advantageous if the reducing agent on the one hand and the optionally functionalized, preferentially optionally fatty acid functionalized, preferably optionally C₅-C₃₄-fatty acid functionalized, especially optionally C₈-C₃₄-fatty acid functionalized, 3-oxobutyric acid polyol esters (I) to be reduced, on the other hand, are used in a molar ratio of reducing agent/keto groups —C(O)— of the acetyl function CH₃— C(O)— of the 3-oxobutyrate radical of the general formula (I′), as defined hereinabove, in a range of from 1:1 to 10:1, especially in a range of from 2:1 to 8:1, preferentially in a range of from 3:1 to 6:1.

Such a complete reduction is particularly advantageous, since no residual reactants, especially in the form of the optionally functionalized, preferentially optionally fatty acid functionalized, preferably optionally C₅-C₃₄-fatty acid functionalized, especially optionally C₈-C₃₄-fatty acid functionalized, 3-oxobutyric acid polyol ester (I) are present in the reduction product obtained, which is why a costly and energy-intensive purification to obtain a pure reaction product is not necessary.

According to an alternative embodiment of the present invention, the reduction may be carried out as a non-complete reduction of all keto groups —C(O)— of the acetyl function CH₃— C(O)— of the at least one 3-oxobutyrate radical of the general formula (I′), as defined hereinabove, to a 3-hydroxybutyrate radical of the general formula (II′), as defined hereinabove. Thus, according to this embodiment, the reduction may be carried out such that not all the keto groups —C(O)— of the acetyl function CH₃— C(O)— of the at least one 3-oxobutyrate radical of the general formula (I′), as defined hereinabove, are converted to a 3-hydroxybutyrate radical of the general formula (II′), as defined hereinabove.

Furthermore, it is preferred in this context if the reduction is carried out such that the reaction product comprises at least one acetyl function CH₃— C(O)—and/or that the reaction product comprises at least one 3-oxobutyrate radical of the general formula (I′), as defined hereinabove.

In this context, it is preferred if the reducing agent is used in molar amounts below the equimolar amount and/or in a substoichiometric amount relative to the keto groups —C(O)— of the acetyl function CH₃— C(O)— of the at least one 3-oxobutyrate radical of the general formula (I′).

In other words, according to this alternative embodiment, the reduction is carried out substoichiometrically by means of at least one reducing agent as defined hereinabove; substoichiometrically in this case means that a molar excess of optionally functionalized, preferentially optionally fatty acid functionalized, preferably optionally C₅-C₃₄-fatty acid functionalized, especially optionally C₈-C₃₄-fatty acid functionalized, 3-oxobutyric acid polyol ester (I), especially 3-oxobutyric acid polygylcerol ester, is used, or a molar deficiency (i. e. a molar shortage so to speak) of reducing agent is used, so that not all keto groups of the 3-oxobutyrate radical of the general formula (I′), as defined hereinabove, are converted to hydroxyl groups as present in the 3-hydroxybutyrate radical of the general formula (II′), as defined hereinabove.

Such incomplete reduction can be particularly advantageous, since the presence of both 3-oxobutyrate radicals of the general formula (I′) and 3-hydroxybutyrate radicals of the general formula (II′) means that the reaction product exhibits a retardation effect, i. e. due to the different rates of degradation of the various radicals, there is a different rate of availability or release of the active ingredient 3-hydroxybutyric acid. Overall, therefore, a longer-term availability or release is possible and thus a sustained-release therapy with 3-hydroxybutyric acid is possible.

According to another particular embodiment of the method according to the invention, the reaction product obtained can be fractionated after the reduction has been carried out, especially fractionated by distillation.

It is also preferred in the context of the present invention if unreacted starting compounds are separated from the reaction product and subsequently recycled. In this context, the starting compounds are the optionally functionalized, preferentially optionally fatty acid functionalized, preferably optionally C₅-C₃₄-fatty acid functionalized, especially optionally C₈-C₃₄-fatty acid functionalized, 3-oxobutyric acid polyol ester (I), especially 3-oxobutyric acid polyglycerol ester, and optionally the reducing agent used according to the invention.

With regard to the optionally functionalized, preferentially optionally fatty acid functionalized, preferably optionally C₅-C₃₄-fatty acid functionalized, especially optionally C₈-C₃₄-fatty acid functionalized, 3-oxobutyric acid polyol ester (I), especially 3-oxobutyric acid polyglycerol ester, used as a starting compound, it is especially preferred if it corresponds to the general formula (Ia)

(R¹O)ₘ—(X)—(OR¹)ₙ (Ia)

wherein, in the general formula (Ia),
X represents an organic radical, especially a preferentially saturated organic radical comprising 3 to 21 carbon atoms, preferentially 4 to 21 carbon atoms, and optionally comprising 1 to 9 oxygen atoms, preferentially selected from an alkyl radical or a (poly)alkyl ether radical, especially (poly)alkylene glycol radical, more preferably selected from C₃-C₂₁-alkyl radical, preferentially C₄-C₂₁-alkyl radical, or C₃-C₂₁-(poly)alkyl ether radical, preferentially C₄-C₂₁-(poly)alkyl ether radical, especially C₃-C₂₁-(poly)alkylene glycol radical, preferentially C₄-C₂₁-(poly)alkylene glycol radical,
the variables m and n, each independently of one another, represent an integer from 1 to 10,
the radical R¹, each independently of one another, identical or different, represents: hydrogen, a radical CH₃— C(O)—CH₂— C(O)—or a radical R², wherein the radical R² represents a radical of the type linear (straight-chained) or branched, saturated or mono- or polyunsaturated (C₁-C₃₃-alkyl)-C(O)—, especially (C₄-C₃₃-alkyl)-C(O)—, preferably (C₇-C₃₃-alkyl)-C(O)—, however, with the proviso that at least one radical R¹, especially at least two radicals R¹, does not represent hydrogen, ad with the proviso that at least one radical R¹, especially at least two radicals R¹, represents a radical CH₃— C(O)—CH₂— C(O)—;
   especially wherein the groups R¹O—are in any position of the radical X, preferentially wherein at least one group R¹O—is terminal

According to a particular embodiment of the method according to the invention, the optionally functionalized, preferentially optionally fatty acid functionalized, preferably optionally C₅-C₃₄-fatty acid functionalized, especially optionally C₈-C₃₄-fatty acid functionalized, 3-oxobutyric acid polyol ester (I), especially 3-oxobutyric acid polyglycerol ester, used as a starting compound, may correspond to the general formula (Ib)

R¹O—CH₂—CH(OR¹)—CH₂—[O—CH₂—CH(OR¹)—CH₂]ₚ—OR¹ (Ib)

wherein, in the general formula (Ib),
the variable p represents an integer from 0 to 6, especially from 1 to 4, preferentially 1 or 2, more preferably 1,
the radical R¹, each independently of one another, identical or different, represents: hydrogen, CH₃—C(O)—CH₂— C(O)—or a radical R², wherein the radical R² represents a radical of the type linear (straight-chained) or branched, saturated or mono- or polyunsaturated (C₁-C₃₃-alkyl)-C(O)—, especially (C₄-C₃₃-alkyl)-C(O)—, preferably (C₇-C₃₃-alkyl)-C(O)—, however, with the proviso that at least one radical R¹, especially at least two radicals R¹, does not represent hydrogen, and with the proviso that at least one radical R¹, especially at least two radicals R¹, represents a radical CH₃— C(O)—CH₂— C(O)—.

According to a further particular embodiment of the method according to the invention, the optionally functionalized, preferentially optionally fatty acid functionalized, preferably optionally C₅-C₃₄-fatty acid functionalized, especially optionally C₈-C₃₄-fatty acid functionalized, 3-oxobutyric acid polyol ester (I), especially 3-oxobutyric acid polyglycerol ester, used as a starting compound, may correspond to the general formula (Ic)

R¹O—CH₂—CH(OR¹)—CH₂—O—CH₂—CH(OR¹)—CH₂—OR¹ (Ic)

wherein, in the general formula (Ic), the radical R¹, each independently of one another, identical or different, represents: hydrogen, CH₃— C(O)—CH₂— C(O)—or a radical R², wherein the radical R² represents a radical of the type linear (straight-chained) or branched, saturated or mono- or polyunsaturated (C₁-C₃₃-alkyl)-C(O)—, especially (C₄-C₃₃-alkyl)-C(O)—, preferably (C₇-C₃₃-alkyl)-C(O)—, however, with the proviso that at least one radical R¹, especially at least two radicals R¹, does not represent hydrogen, and with the proviso that at least one radical R¹, especially at least two radicals R¹, represents a radical CH₃— C(O)—CH₂— C(O)—.

According to a further particular embodiment of the method according to the invention, the optionally functionalized, preferentially optionally fatty acid functionalized, preferably optionally C₅-C₃₄-fatty acid functionalized, especially optionally C₅-C₃₄-fatty acid functionalized, 3-oxobutyric acid polyol ester (I), especially 3-oxobutyric acid polyglycerol ester, used as a starting compound, may correspond to the general formula (Id)

R¹O—CH₂—CH(OR¹)—CH₂—OR¹ (Id)

wherein, in the general formula (Id), the radical R¹, each independently of one another, identical or different, represents: hydrogen, CH₃— C(O)—CH₂— C(O)—or a radical R², wherein the radical R² represents a radical of the type linear (straight-chained) or branched, saturated or mono- or polyunsaturated (C₁-C₃₃-alkyl)-C(O)—, especially (C₈-C₃₃-alkyl)-C(O)—, preferably (C₇-C₃₃-alkyl)-C(O)—, however, with the proviso that at least one radical R¹, especially at least two radicals R¹, does not represent hydrogen, and with the proviso that at least one radical R¹, especially at least two radicals R¹, represents a radical CH₃— C(O)—CH₂— C(O)—.

Especially, the optionally functionalized, preferentially optionally fatty acid functionalized, preferably optionally C₅-C₃₄-fatty acid functionalized, especially optionally C₅-C₃₄-fatty acid functionalized, 3-oxobutyric acid polyol ester (I), especially 3-oxobutyric acid polyglycerol ester, used as a starting compound, may be obtainable and/or may be obtained by a synthesis route (A),

wherein, according to synthesis route (A), in a first method step, at least one compound of the general formula (III)

CH₃—C(O)—CH₂—C(O)OR³ (III)

wherein, in the general formula (III), the radical R³ represents C₁-C₄-alkyl, especially methyl or ethyl, preferably ethyl,
is reacted with at least one polyol (IV) comprising at least two hydroxyl groups (OH-groups), especially polyglycerol,
optionally followed by a second method step, especially in the case that the 3-oxobutyric acid polyol ester (I), especially the 3-oxobutyric acid polyglycerol ester, is functionalized, preferentially fatty acid functionalized, preferably C₅-C₃₄-fatty acid functionalized, especially C₈-C₃₄-fatty acid functionalized, wherein the second method step comprises
(i) an at least partial functionalization, especially an at least partial esterification, of hydroxyl groups still present by means of at least one carboxylic acid and/or its ester or anhydride, preferentially by means of at least one fatty acid and/or its ester or anhydride, preferably by means of at least one C₅-C₃₄-fatty acid and/or its ester or anhydride, especially by means of at least one C₈-C₃₄-fatty acid and/or its ester or anhydride, and/or
(ii) a partial transesterification of ester groups introduced in the first method step by means of at least one carboxylic acid and/or ester thereof, preferentially by means of at least one fatty acid and/or ester thereof, preferably by means of at least one C₅-C₃₄-fatty acid and/or ester thereof, especially by means of at least one C₈-C₃₄-fatty acid and/or ester thereof

Similarly, in the case that the 3-oxobutyric acid polyol ester (I) used as a starting compound, as defined hereinabove, is present in functionalized, preferentially fatty acid functionalized, preferably C₅-C₃₄-fatty acid functionalized, more preferably C₈-C₃₄-fatty acid functionalized, form, the functionalized, preferentially fatty acid functionalized, preferably C₅-C₃₄-fatty acid functionalized, more preferably C₈-C₃₄-fatty acid functionalized, 3-oxobutyric acid polyol ester (I), especially 3-oxobutyric acid polyglycerol ester, used as a starting compound, may be obtainable and/or may be obtained by one of the two following synthesis routes (A) or (B),
(A) wherein, according to a (first) synthesis route (A), first of all in a first method step at least one compound of the general formula (III)

CH₃—C(O)—CH₂—C(O)OR³ (III)
wherein, in the general formula (III), the radical R³ represents C₁-C₄-alkyl, especially methyl or ethyl, preferably ethyl,
is reacted with at least one polyol (IV) comprising at least two hydroxyl groups (OH-groups), especially polyglycerol,
followed by a second method step, wherein the second method step comprises
(i) an at least partial functionalization, especially an at least partial esterification, of hydroxyl groups still present by means of at least one carboxylic acid and/or its ester or anhydride, preferentially by means of at least one fatty acid and/or its ester or anhydride, preferably by means of at least one C₅-C₃₄-fatty acid and/or its ester or anhydride, especially by means of at least one C₈-C₃₄-fatty acid and/or its ester or anhydride, and/or
(ii) a partial transesterification of ester groups introduced in the first method step by means of at least one carboxylic acid and/or its ester, preferentially by means of at least one fatty acid and/or its ester, preferably by means of at least one C₅-C₃₄-fatty acid and/or its ester, especially by means of at least one C₅-C₃₄-fatty acid and/or its ester,
or else
(B) wherein according to a (second, alternative to (A)) synthesis route (B), first of all in a first method step at least one polyol (IV) comprising at least two hydroxyl groups (OH-groups), especially polyglycerol,
   is reacted with at least one carboxylic acid and/or its ester or anhydride, preferentially with at least one fatty acid and/or its ester or anhydride, preferably with at least one C₅-C₃₄-fatty acid and/or its ester or anhydride, especially with at least one C₈-C₃₄-fatty acid and/or its ester or anhydride,
   followed by a second method step, wherein the second method step comprises
   (i) an at least partial esterification of hydroxyl groups still present by means of a compound of the general formula (III), as defined hereinabove, and/or
   (ii) a partial transesterification of ester groups introduced in the first method step by means of a compound of the general formula (III), as defined hereinabove.

With regard to the polyol (IV) used in the method according to the invention, it is particularly preferred if the polyol (IV) corresponds to the general formula (IVa)

(HO)ₘ—(X)—(OH)ₙ (IVa)

wherein, in the general formula (IVa),
X represents an organic radical, especially a preferentially saturated organic radical comprising 3 to 21 carbon atoms, preferentially 4 to 21 carbon atoms, and optionally comprising 1 to 9 oxygen atoms, preferentially selected from an alkyl radical or a (poly)alkyl ether radical, especially (poly)alkylene glycol radical, more preferably selected from C₃-C₂₁-alkyl radical, preferentially C₄-C₂₁-alkyl radical, or C₃-C₂₁-(poly)alkyl ether radical, preferentially C₄-C₂₁-(poly)alkyl ether radical, especially C₃-C₂₁-(poly)alkylene glycol radical, preferentially C₄-C₂₁-(poly)alkylene glycol radical,
the variables m and n, each independently of one another, represent an integer from 1 to 10;
   especially wherein the hydroxyl groups of the polyol (IV) are in any position of the radical X, preferentially wherein at least one hydroxyl group is terminal and/or is a primary hydroxyl group.

Furthermore, it may also be preferred if the polyol (IV) is a polyglycerol of the general formula (IVb)

HO—CH₂—CH(OH)—CH₂—[O—CH₂—CH(OH)—CH₂]ₚ—OH (IVb)

wherein, in the general formula (IVb), the variable p represents an integer from 0 to 6, especially from 1 to 4, preferentially 1 or 2, more preferably 1.

Furthermore, according to the method of the invention, it may be preferred if the polyol (IV) is a diglycerol of formula (IVc)

HO—CH₂—CH(OH)—CH₂—O—CH₂—CH(OH)—CH₂—OH (IVc)

Furthermore, according to the method of the invention, it may be provided that the polyol (IV) is not propane-1,2,3-triol (glycerol).

Alternatively, according to the method of the invention, it may be provided that the polyol (IV) is propane-1,2,3-triol (glycerol).

Furthermore, according to the method of the invention, it may be provided that the polyol (IV) is selected from alkanediols, especially C₃-C₂₁-alkanediols, preferentially C₄-C₂₁-alkanediols, preferably linear or branched alkanediols, more preferably linear or branched C₃-C₂₁-alkanediols, preferentially C₄-C₂₁-alkanediols, even more preferably linear C₃-C₂₁-alkanediols, preferentially C₄-C₂₁-alkanediols, more preferably linear C₃-C₂-alkanediols, preferentially C₄-C₂₁-alkanediols, with at least one terminal and/or primary hydroxyl group, yet further preferred pentanediol, especially 1,2-pentanediol.

In the method according to the invention, it is preferred if the carboxylic acid and/or its ester, preferentially the fatty acid and/or the fatty acid ester, is a carboxylic acid and/or a carboxylic acid ester of the general formula (V)

R²—O—R⁴ (V)

wherein, in the general formula (V),
the radical R² represents a radical of the type linear (straight-chained) or branched, saturated or mono- or polyunsaturated (C₁-C₃₃-alkyl)-C(O)—type, especially (C₄-C₃₃-alkyl)-C(O)—, preferably (C₇-C₃₃-alkyl)-C(O)—,
the radical R⁴ represents hydrogen or C₁-C₄-alkyl, especially hydrogen, methyl or ethyl, more preferably hydrogen.

Furthermore, it is preferred in the method according to the invention if the carboxylic acid anhydride, preferentially the fatty acid anhydride, is a carboxylic acid anhydride of the general formula (VI)

R²—O—R² (VI)

wherein, in the general formula (VI), the radical R² represents a radical of the type linear (straight-chained) or branched, saturated or mono- or polyunsaturated (C₁-C₃₃-alkyl)-C(O)— type, especially (C₄-C₃₃-alkyl)-C(O)—, preferably (C₇-C₃₃-alkyl)-C(O)—.

Following the reduction, hydroxyl groups still present in the reaction product (II) after the reduction has been carried out can be at least partially, preferentially completely, functionalized, especially esterified.

According to a particular embodiment of the production method according to the invention, the reduction can thus be followed by a partial, especially complete, functionalization, especially esterification, of hydroxyl groups still present.

Especially, it may be provided in the production method according to the invention that ester groups present in the reaction product (II) after the reduction has been carried out, especially in the form of 3-hydroxybutyrate radicals according to the general formula (II′) as defined hereinabove, are partially transesterified.

According to the invention, it may be especially provided that the reduction is followed by a partial transesterification of ester groups present, especially in the form of 3-hydroxybutyrate radicals according to the general formula (II′), as defined hereinabove.

In this context, it is particularly preferred if the functionalization and/or transesterification is carried out by means of a fatty acid, preferentially a C₅-C₃₄-fatty acid, preferably a C₈-C₃₄-fatty acid, especially in free form or in the form of its ester or anhydride, especially as defined hereinabove.

According to a particular embodiment of the method according to the invention, the fatty acid, preferentially the C₅-C₃₄-fatty acid, preferably the C₈-C₃₄-fatty acid, especially in free form or in the form of its ester or anhydride, may be selected from the group of caprylic acid, pelargonic acid, capric acid, undecanoic acid, lauric acid, tridecanoic acid, myristic acid, pentadecanoic acid, palmitic acid, margaric acid, stearic acid, nonadecanoic acid, arachidic acid, heneicosanoic acid, behenic acid, lignoceric acid, cerotinic acid, montanic acid, melissic acid, laccic acid, geddic acid, undecylic acid, myristoleic acid, palmitoleic acid, margaroleic acid, petroselinic acid, oleic acid, elaidic acid, vaccenic acid, gadoleic acid, cetoleic acid, erucic acid, nervonic acid, linoleic acid, linolenic acids, calendulic acid, punicic acid, eleostearic acids, stearidonic acid, arachidonic acid, eicosapentaenoic acid, docosadienoic acid, docosatetraenoic acid, docosapentaenoic acid, docosahexaenoic acid, and tetracosahexaenoic acid, as well as mixtures thereof.

According to another particular embodiment of the method according to the invention, the fatty acid, preferentially the C₅-C₃₄-fatty acid, preferably the C₈-C₃₄-fatty acid, especially in free form or in the form of its ester or anhydride, may be selected from the group of myristic acid, pentadecanoic acid, palmitoleic acid, cetoleic acid, oleic acid, gadoleic acid, cetoleic acid, erucic acid, arachidonic acid, eicosapentaenoic acid, docosadienoic acid, docosatetraenoic acid, docosapentaenoic acid, docosahexaenoic acid, tetracosahexaenoic acid as well as mixtures thereof, preferentially eicosapentaenoic acid and docosahexaenoic acid as well as mixtures thereof.

Furthermore, according to a particular embodiment of the method according to the invention, the fatty acid, preferentially the C₅-C₃₄-fatty acid, preferably the C₈-C₃₄-fatty acid, especially in free form or in the form of its ester or anhydride, may be selected from the group of fatty acids based on fish oil and/or occurring in fish oils, especially eicosapentaenoic acid, docosadienoic acid, docosatetraenoic acid, docosapentaenoic acid, docosahexaenoic acid and tetracosahexaenoic acid as well as mixtures thereof, preferentially eicosapentaenoic acid, docosahexaenoic acid as well as mixtures thereof.

Especially, in the production method according to the invention, during the reduction, the position and number of the hydroxyl groups —CH(OH)—of the 3-hydroxybutyrate radical of the general formula (II′), as defined hereinabove, obtained by reduction is not changed compared to the keto groups —C(O)— of the 3-oxobutyrate radical of the general formula (I′), as defined hereinabove, to be reduced.

In the context of the method according to the invention, it is preferred if the reduction of the at least one 3-oxobutyrate radical of the general formula (I′), as defined hereinabove, selectively takes place only at the keto group —C(O)— of the acetyl function CH₃— C(O)— of the 3-oxobutyrate radical (I′), as defined hereinabove; especially wherein no further reactions take place. In this context, it is preferred if, during the reduction, no side reactions, especially no rearrangements, cleavages or additions, take place.

In the method according to the invention, as a reaction product, one or more optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol esters (II), especially 3-hydroxybutyric acid polyglycerol esters, of the general formula (IIa)

(R⁵O)ₘ—(X)—(OR⁵)ₙ (IIa)

may be obtained,
wherein, in the general formula (IIa),
X represents an organic radical, especially a preferentially saturated organic radical comprising 3 to 21 carbon atoms, preferentially 4 to 21 carbon atoms, and optionally comprising 1 to 9 oxygen atoms, preferentially selected from an alkyl radical or a (poly)alkyl ether radical, especially (poly)alkylene glycol radical, more preferably selected from C₃-C₂₁-alkyl radical, preferentially C₄-C₂₁-alkyl radical, or C₃-C₂₁-(poly)alkyl ether radical, preferentially C₄-C₂₁-(poly)alkyl ether radical, especially C₃-C₂₁-(poly)alkylene glycol radical, preferentially C₄-C₂₁-(poly)alkylene glycol radical,
the variables m and n, each independently of one another, represent an integer from 1 to 10,
the radical R⁵, each independently of one another, identical or different, represents: hydrogen, a radical R², wherein the radical R² represents a radical of the type linear (straight-chained) or branched, saturated or mono- or polyunsaturated (C₁-C₃₃-alkyl)-C(O)—, especially (C₄-C₃₃-alkyl)-C(O)—, preferably (C₇-C₃₃-alkyl)-C(O)—, or a radical CH₃— CH(OR⁶)—CH₂— C(O)—, wherein the radical R⁶ represents hydrogen or a radical R², as defined hereinabove, however, with the proviso that at least one radical R⁵, especially at least two radicals R⁵, does not represent hydrogen, and with the proviso that at least one radical R⁵, especially at least two radicals R⁵, represents a radical CH₃— CH(OR⁶)—CH₂— C(O)—,
   however, with the proviso that the number and position of the 3-hydroxybutyrate radicals of the general formula (II′) in the 3-hydroxybutyric acid polyol ester (II) obtained by the reduction correspond to the number and position of the 3-oxobutyrate radicals of the general formula (I′) in the 3-oxobutyric acid polyol ester (I) to be reduced.

Especially, the groups R⁵O—may be in any position of the radical X, preferentially wherein at least one group R⁵O— is terminal

Thus, the term “functionalized” or “optionally functionalized” means that free hydroxyl groups (OH-groups), which may be present in the polyol (IV) as well as in the 3-hydroxybutyrate radical of the general formula (II′), may optionally be functionalized.

According to the invention, it is envisaged that the hydroxyl groups (OH-groups) of the 3-hydroxybutyrate radical of general formula (II′) formed by the reduction have the same position in the 3-hydroxybutyric acid polyol ester (II) as the keto groups—C(O)— of the acetyl function CH3—C(O)—in the 3-oxobutyrate radical of general formula (I′) before the reaction. In this context, the number of hydroxyl groups generated by reduction corresponds to the number of reduced keto groups.

Especially, as a reaction product, one or more optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol esters (II), especially 3-hydroxybutyric acid polyglycerol esters, of the general formula (IIb)

R⁵O—CH₂—CH(OR⁵)—CH₂—[O—CH₂—CH(OR⁵)—CH₂]ₚ—OR⁵ (IIb)

may be obtained,
wherein, in the general formula (IIb),
the variable p represents an integer from 0 to 6, especially from 1 to 4, preferentially 1 or 2, more preferably 1,
the radical R⁵, each independently of one another, identical or different, represents: hydrogen, a radical R², wherein the radical R² represents a radical of the type linear (straight-chained) or branched, saturated or mono- or polyunsaturated (C₁-C₃₃-alkyl)-C(O)—, especially (C₄-C₃₃-alkyl)-C(O)—, preferably (C₇-C₃₃-alkyl)-C(O)—, or a radical CH₃— CH(OR⁶)—CH₂— C(O)—, wherein the radical R⁶ represents hydrogen or a radical R², as defined hereinabove, however, with the proviso that at least one radical R⁵, especially at least two radicals R⁵, does not represent hydrogen, and with the proviso that at least one radical R⁵, especially at least two radicals R⁵, represents a radical CH₃— CH(OR⁶)—CH₂— C(O)—,
   however, with the proviso that the number and position of the 3-hydroxybutyrate radicals of the general formula (II′) in the 3-hydroxybutyric acid polyol ester (II) obtained by the reduction correspond to the number and position of the 3-oxobutyrate radicals of the general formula (I′) in the 3-oxobutyric acid polyol ester (I) to be reduced.

According to a particular embodiment of the method according to the invention, as a reaction product, one or more optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol esters (II), especially 3-hydroxybutyric acid polyglycerol esters, of the general formula (IIc)

R⁵O—CH₂—CH(OR⁵)—CH₂—O—CH₂—CH(OR⁵)—CH₂—OR⁵ (IIc)

may be obtained,
wherein, in the general formula (IIc), the radical R⁵, each independently of one another, identical or different, represents: hydrogen, a radical R², wherein the radical R² represents a radical of the type linear (straight-chained) or branched, saturated or mono- or polyunsaturated (C₁-C₃₃-alkyl)-C(O)—, especially (C₄-C₃₃-alkyl)-C(O)—, preferably (C₇-C₃₃-alkyl)-C(O)—, or a radical CH₃— CH(OR⁶)—CH₂— C(O)—, wherein the radical R⁶ represents hydrogen or a radical R², as defined hereinabove, however, with the proviso that at least one radical R⁵, especially at least two radicals R⁵, does not represent hydrogen, and with the proviso that at least one radical R⁵, especially at least two radicals R⁵, represents a radical CH₃— CH(OR⁶)—CH₂— C(O)—,
however, with the proviso that the number and position of the 3-hydroxybutyrate radicals of the general formula (II′) in the 3-hydroxybutyric acid polyol ester (II) obtained by the reduction correspond to the number and position of the 3-oxobutyrate radicals of the general formula (I′) in the 3-oxobutyric acid polyol ester (I) to be reduced.

According to a further particular embodiment of the method according to the invention, as a reaction product, one or more optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol esters (II), especially 3-hydroxybutyric acid polyglycerol esters, of the general formula (IId)

R⁵O—CH₂—CH(OR⁵)—CH₂—OR⁵ (IId)

may be obtained,
wherein, in the general formula (IId), the radical R⁵, each independently of one another, identical or different, represents: hydrogen, a radical R², wherein the radical R² represents a radical of the type linear (straight-chained) or branched, saturated or mono- or polyunsaturated (C₁-C₃₃-alkyl)-C(O)—, especially (C₈-C₃₃-alkyl)-C(O)—, preferably (C₇-C₃₃-alkyl)-C(O)—, or a radical CH₃— CH(OR⁶)—CH₂— C(O)—, wherein the radical R⁶ represents hydrogen or a radical R², as defined hereinabove, however, with the proviso that at least one radical R⁵, especially at least two radicals R⁵, does not represent hydrogen, and with the proviso that at least one radical R⁵, especially at least two radicals R⁵, represents a radical CH₃— CH(OR⁶)—CH₂— C(O)—,
however, with the proviso that the number and position of the 3-hydroxybutyrate radicals of the general formula (II′) in the 3-hydroxybutyric acid polyol ester (II) obtained by the reduction correspond to the number and position of the 3-oxobutyrate radicals of the general formula (I′) in the 3-oxobutyric acid polyol ester (I) to be reduced.

According to a further embodiment of the method according to the invention, as a reaction product, a mixture of at least two different optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol esters (II), especially 3-hydroxybutyric acid polyglycerol esters, as defined hereinabove, may be obtained, however, with the proviso that the number and position of the 3-hydroxybutyrate radicals of the general formula (II′) in the 3-hydroxybutyric acid polyol ester (II) obtained by the reduction correspond to the number and position of the 3-oxobutyrate radicals of the general formula (I′) in the 3-oxobutyric acid polyol ester (I) to be reduced.

According to a further particular embodiment of the method according to the invention, as a reaction product, a mixture of at least three different optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol esters (II), especially 3-hydroxybutyric acid polyglycerol esters, as defined hereinabove, may be obtained, however, with the proviso that the number and position of the 3-hydroxybutyrate radicals of the general formula (II′) in the 3-hydroxybutyric acid polyol ester (II) obtained by the reduction correspond to the number and position of the 3-oxobutyrate radicals of the general formula (I′) in the 3-oxobutyric acid polyol ester (I) to be reduced.

As previously stated, it may be provided according to the invention that in the reaction product, the 3-hydroxybutyrate radical of the general formula (II′), as defined hereinabove, is present in the form of the (R)-configured enantiomer.

In this context, the chirality center (asymmetric carbon atom) is located at the 3-position of the 3-hydroxybutyrate radical of the general formula (II′).

Furthermore, as a reaction product, one or more 3-hydroxybutyric acid polyol esters (II″), especially 3-hydroxybutyric acid polyglycerol esters, of the general formula (IIa″)

(R⁷O)ₘ—(X)—(OR⁷)ₙ (IIa″)

may be obtained,
wherein, in the general formula (IIa″),
X represents an organic radical, especially a preferentially saturated organic radical comprising 3 to 21 carbon atoms, preferentially 4 to 21 carbon atoms, and optionally comprising 1 to 9 oxygen atoms, preferentially selected from an alkyl radical or a (poly)alkyl ether radical, especially (poly)alkylene glycol radical, more preferably selected from C₃-C₂₁-alkyl radical, preferentially C₄-C₂₁-alkyl radical, or C₃-C₂₁-(poly)alkyl ether radical, preferentially C₄-C₂₁-(poly)alkyl ether radical, especially C₃-C₂₁-(poly)alkylene glycol radical, preferentially C₄-C₂₁-(poly)alkylene glycol radical,
the variables m and n, each independently of one another, represent an integer from 1 to 10,
the radical R⁷, each independently of one another, identical or different, represents: hydrogen or a radical CH₃— CH(OH)—CH₂— C(O)—, however, with the proviso that at least one radical R⁷, especially at least two radicals R¹, does not represent hydrogen,
   however, with the proviso that the number and position of the 3-hydroxybutyrate radicals of the general formula (II″) in the 3 hydroxybutyric acid polyol ester (II) obtained by the reduction correspond to the number and position of the 3-oxobutyrate radicals of the general formula (I′) in the 3-oxobutyric acid polyol ester (I) to be reduced;

Especially, it is preferred if the groups R⁷O—are in any position of the radical X, preferentially at least one group R⁷O—should be terminal.

Furthermore, in the production method according to the invention, as a reaction product, one or more 3-hydroxybutyric acid polyol esters (II″), especially 3-hydroxybutyric acid polyglycerol esters, of the general formula (IIb″)

R⁷O—CH₂—CH(OR⁷)—CH₂—[O—CH₂—CH(OR⁷)—CH₂]ₚ—OR⁷ (IIb″)

may be obtained,
wherein, in the general formula (IIb″),
the variable p represents an integer from 0 to 6, especially from 1 to 4, preferentially 1 or 2, more preferably 1,
the radical R⁷, each independently of one another, identical or different, represents: hydrogen or CH₃— CH(OH)—CH₂— C(O)—, however, with the proviso that at least one radical R⁷, especially at least two radicals R⁷, does not represent hydrogen,
   however, with the proviso that the number and position of the 3-hydroxybutyrate radicals of the general formula (II′) in the 3-hydroxybutyric acid polyol ester (II) obtained by the reduction correspond to the number and position of the 3-oxobutyrate radicals of the general formula (I′) in the 3-oxobutyric acid polyol ester (I) to be reduced.

Moreover, in the method according to the invention, as a reaction product, one or more 3-hydroxybutyric acid polyglycerol esters (II″) of the general formula (IIc″)

R¹O—CH₂—CH(OR⁷)—CH₂—O—CH₂—CH(OR⁷)—CH₂—OR⁷ (IIc″)

may be obtained,
wherein, in the general formula (IIc″), the radical R⁷, each independently of one another, identical or different, represents hydrogen or CH₃— CH(OH)—CH₂— C(O)—, however, with the proviso that at least one radical R⁷, especially at least two radicals R⁷, does not represent hydrogen,
however, with the proviso that the number and position of the 3-hydroxybutyrate radicals of the general formula (II′) in the 3-hydroxybutyric acid polyol ester (II) obtained by the reduction correspond to the number and position of the 3-oxobutyrate radicals of the general formula (I′) in the 3-oxobutyric acid polyol ester (I) to be reduced.

Moreover, in the method according to the invention, as a reaction product, one or more 3-hydroxybutyric acid polyol esters (II″), especially 3-hydroxybutyric acid polyglycerol esters, of the general formula (IId″)

R¹O—CH₂—CH(OR⁷)—CH₂—OR⁷ (IId″)

may be obtained,
wherein, in the general formula (IId″) the radical R¹, each independently of one another, identical or different, represents hydrogen or CH₃— CH(OH)—CH₂— C(O)—, however, with the proviso that at least one radical R⁷, especially at least two radicals R⁷, does not represent hydrogen,
however, with the proviso that the number and position of the 3-hydroxybutyrate radicals of the general formula (II′) in the 3-hydroxybutyric acid polyol ester (II) obtained by the reduction correspond to the number and position of the 3-oxobutyrate radicals of the general formula (I′) in the 3-oxobutyric acid polyol ester (I) to be reduced.

According to a particular embodiment of the production method according to the invention, as a reaction product, a mixture of at least two different 3-hydroxybutyric acid polyol esters (II″), especially 3-hydroxybutyric acid polyglycerol esters, especially as defined hereinabove, may be obtained, however, with the proviso, that the number and position of the 3-hydroxybutyrate radicals of the general formula (II′) in the 3-hydroxybutyric acid polyol ester (II) obtained by the reduction correspond to the number and position of the 3-oxobutyrate radicals of the general formula (I′) in the 3-oxobutyric acid polyol ester (I) to be reduced.

According to a further particular embodiment of the method according to the invention, as a reaction product, a mixture of at least three different 3-hydroxybutyric acid polyol esters (II″), especially 3-hydroxybutyric acid polyglycerol esters, especially as defined hereinabove, may be obtained, however, with the proviso, that the number and position of the 3-hydroxybutyrate radicals of the general formula (II′) in the 3-hydroxybutyric acid polyol ester (II) obtained by the reduction correspond to the number and position of the 3-oxobutyrate radicals of the general formula (I′) in the 3-oxobutyric acid polyol ester (I) to be reduced.

Especially, it may be provided in the context of the present invention that in the reaction product, the 3-hydroxybutyrate radical of the general formula (II′), as defined hereinabove, is present in the form of the (R)-configured enantiomer.

In this context the chirality center (asymmetric carbon atom) is located at the 3-position of the 3-hydroxybutyrate radical of the general formula (II′).

According to a further embodiment of the present invention, as a reaction product, one or more functionalized, especially fatty acid functionalized, preferably C₅-C₃₄-fatty acid functionalized, more preferably C₅-C₃₄-fatty acid functionalized, 3-hydroxybutyric acid polyol ester (II″′), especially 3-hydroxybutyric acid polyglycerol ester, of the general formula (IIa′″)

(R⁵O)ₘ—(X)—(OR⁵)ₙ (IIa″′)

may be obtained,
wherein, in the general formula (IIa″),
X represents an organic radical, especially a preferentially saturated organic radical comprising 3 to 21 carbon atoms, preferentially 4 to 21 carbon atoms, and optionally comprising 1 to 9 oxygen atoms, preferentially selected from an alkyl radical or a (poly)alkyl ether radical, especially (poly)alkylene glycol radical, more preferably selected from C₃-C₂₁-alkyl radical, preferentially C₄-C₂₁-alkyl radical, or C₃-C₂₁-(poly)alkyl ether radical, preferentially C₄-C₂₁-(poly)alkyl ether radical, especially C₃-C₂₁-(poly)alkylene glycol radical, preferentially C₄-C₂₁-(poly)alkylene glycol radical,
the variables m and n, each independently of one another, represent an integer from 1 to 10,
the radical R⁵, each independently of one another, identical or different, represents: hydrogen, a radical R², wherein the radical R² represents a radical of the type linear (straight-chained) or branched, saturated or mono- or polyunsaturated (C₁-C₃₃-alkyl)-C(O)—, especially (C₈-C₃₄-alkyl)-C(O)—, preferably (C₇-C₃₃-alkyl)-C(O)—, or a radical CH₃— CH(OR⁶)—CH₂— C(O)—, wherein the radical R⁶ represents hydrogen or a radical R², as defined hereinabove, however, with the proviso that at least one radical R⁵, especially at least two radicals R⁵, does not represent hydrogen, and with the proviso that at least one radical R⁵, especially at least two radicals R⁵, represents a radical CH₃— CH(OR⁶)—CH₂— C(O)—, and with the proviso that at least one radical R⁵ and/or one radical R⁶ represents a radical R²,
   however, with the proviso that the number and position of the 3-hydroxybutyrate radicals of the general formula (II′) in the 3-hydroxybutyric acid polyol ester (II) obtained by the reduction correspond to the number and position of the 3-oxobutyrate radicals of the general formula (I′) in the 3-oxobutyric acid polyol ester (I) to be reduced.

Especially, the groups R⁵O—may be in any position of the radical X, preferentially wherein at least one group R⁵O—is terminal.

Especially, according to a particular embodiment, in the general formula (IIa″), the radical R⁵, each independently of one another, identical or different, may represent: a radical CH₃— CH(OR⁶)—CH₂— C(O)—, wherein the radical R⁶ represents a radical R², as defined hereinabove, or else a radical R², as defined hereinabove, however, with the proviso that at least one radical R⁵, especially at least two radicals R⁵, represents a radical CH₃— CH(OR⁶)—CH₂— C(O)—, wherein the radical R⁶ comprises a radical R², as defined hereinabove.

Furthermore, according to another particular embodiment, in the general formula (IIa″), the radicals R⁵ and R⁶, each independently of one another, identical or different, may not represent hydrogen.

Especially, in the production method according to the invention, as a reaction product, one or more functionalized, especially fatty acid functionalized, preferably C₅-C₃₄-fatty acid functionalized, more preferably C₈-C₃₄-fatty acid functionalized, 3-hydroxybutyric acid polyol ester (II″′), especially 3-hydroxybutyric acid polyglycerol ester, of the general formula (IIb″′)

R⁵O—CH₂—CH(OR⁵)—CH₂—[O—CH₂—CH(OR⁵)—CH₂]ₚ—OR⁵ (IIb″′)

may be obtained,
wherein, in the general formula (IIb″),
the variable p represents an integer from 0 to 6, especially from 1 to 4, preferentially 1 or 2, more preferably 1,
the radical R⁵, each independently of one another, identical or different, represents: hydrogen, a radical R², wherein the radical R² represents a radical of the type linear (straight-chained) or branched, saturated or mono- or polyunsaturated (C₁-C₃₃-alkyl)-C(O)—, especially (C₄-C₃₃-alkyl)-C(O)—, preferably (C₇-C₃₃-alkyl)-C(O)—, or a radical CH₃— CH(OR⁶)—CH₂— C(O)—, wherein the radical R⁶ represents hydrogen or a radical R², as defined hereinabove, however, with the proviso that at least one radical R⁵, especially at least two radicals R⁵, does not represent hydrogen, and with the proviso that at least one radical R⁵, especially at least two radicals R⁵, represents a radical CH₃— CH(OR⁶)—CH₂— C(O)—, and with the proviso that at least one radical R⁵ and/or one radical R⁶ represents a radical R²,
   however, with the proviso that the number and position of the 3-hydroxybutyrate radicals of the general formula (II′) in the 3-hydroxybutyric acid polyol ester (II) obtained by the reduction correspond to the number and position of the 3-oxobutyrate radicals of the general formula (I′) in the 3-oxobutyric acid polyol ester I) to be reduced.

Especially, according to a particular embodiment, in the general formula (IIb″), the radical R⁵, each independently of one another, identical or different, may represent: a radical CH₃— CH(OR⁶)—CH₂— C(O)—, wherein the radical R⁶ represents a radical R², as defined hereinabove, or else a radical R², as defined hereinabove, however, with the proviso that at least one radical R⁵, especially at least two radicals R⁵, represents a radical CH₃— CH(OR⁶)—CH₂— C(O)—, wherein the radical R⁶ comprises a radical R², as defined hereinabove.

Furthermore, according to an additional particular embodiment, in the general formula (IIb″), R⁵ and R⁶, each independently of one another, identical or different, may not represent hydrogen.

Furthermore, according to a particular embodiment of the method according to the invention, as a reaction product, one or more functionalized, especially fatty acid functionalized, preferably C₅-C₃₄-fatty acid functionalized, more preferably C₈-C₃₄-fatty acid functionalized, 3-hydroxybutyric acid polyol ester (II′), especially 3-hydroxybutyric acid polyglycerol ester, of the general formula (IIc″′)

R⁵O—CH₂—CH(OR⁵)—CH₂—O—CH₂—CH(OR⁵)—CH₂—OR⁵ (IIIc″′)

may be obtained,
wherein, in the general formula (IIc″), the radical R⁵, each independently of one another, identical or different, represents: hydrogen, a radical R², wherein the radical R² represents a radical of the type linear (straight-chained) or branched, saturated or mono- or polyunsaturated (C₁-C₃₃-alkyl)-C(O)—, especially (C₄-C₃₃-alkyl)-C(O)—, preferably (C₇-C₃₃-alkyl)-C(O)—, or a radical CH₃— CH(OR⁶)—CH₂— C(O)—, wherein the radical R⁶ represents hydrogen or a radical R², as defined hereinabove, however, with the proviso that at least one radical R⁵, especially at least two radicals R⁵, does not represent hydrogen, and with the proviso that at least one radical R⁵, especially at least two radicals R⁵, represents a radical CH₃— CH(OR⁶)—CH₂— C(O)—, and with the proviso that at least one radical R⁵ and/or one radical R⁶ represents a radical R², however, with the proviso that the number and position of the 3-hydroxybutyrate radicals of the general formula (II′) in the 3-hydroxybutyric acid polyol ester (II) obtained by the reduction correspond to the number and position of the 3-oxobutyrate radicals of the general formula (I′) in the 3-oxobutyric acid polyol ester (I) to be reduced.

According to a particular embodiment, in the general formula (IIc″), the radical R⁵, each independently of one another, identical or different, may represent: a radical CH₃— CH(OR⁶)—CH₂— C(O)—, wherein the radical R⁶ represents a radical R², as defined hereinabove, or else a radical R², as defined hereinabove, however, with the proviso that at least one radical R⁵, especially at least two radicals R⁵, represents a radical CH₃— CH(OR⁶)—CH₂— C(O)—, wherein the radical R⁶ comprises a radical R², as defined hereinabove.

Furthermore, according to another particular embodiment, in the general formula (IIc″), R⁵ and R⁶, each independently of one another, identical or different, may not represent hydrogen.

Similarly, in the method according to the invention, as a reaction product, one or more functionalized, especially fatty acid functionalized, preferably C₅-C₃₄-fatty acid functionalized, more preferably C₈-C₃₄-fatty acid functionalized, 3-hydroxybutyric acid polyol ester (II″′), especially 3-hydroxybutyric acid polyglycerol ester, of the general formula (IId″′)

R⁵O—CH₂—CH(OR⁵)—CH₂—OR⁵ (IId″′)

may be obtained,
wherein, in the general formula (IId″), the radical R⁵, each independently of one another, identical or different, represents: hydrogen, a radical R², wherein the radical R² represents a radical of the type linear (straight-chained) or branched, saturated or mono- or polyunsaturated (C₁-C₃₃-alkyl)-C(O)—, especially (C₄-C₃₃-alkyl)-C(O)—, preferably (C₇-C₃₃-alkyl)-C(O)—, or a radical CH₃— CH(OR⁶)—CH₂— C(O)—, wherein the radical R⁶ represents hydrogen or a radical R², as defined hereinabove, however, with the proviso that at least one radical R⁵, especially at least two radicals R⁵, does not represent hydrogen, and with the proviso that at least one radical R⁵, especially at least two radicals R⁵, represents a radical CH₃— CH(OR⁶)—CH₂— C(O)—, and with the proviso that at least one radical R⁵ and/or one radical R⁶ represents a radical R²,
however, with the proviso that the number and position of the 3-hydroxybutyrate radicals of the general formula (II′) in the 3-hydroxybutyric acid polyol ester (II) obtained by the reduction correspond to the number and position of the 3-oxobutyrate radicals of the general formula (I′) in the 3-oxobutyric acid polyol ester (I) to be reduced.

Especially, according to a particular embodiment, in the aforementioned general formula (IId″), the radical R⁵, each independently of one another, identical or different, may represent: a radical CH₃— CH(OR⁶)—CH₂— C(O)—, wherein the radical R⁶ represents a radical R², as defined hereinabove, or else a radical R², as defined hereinabove, however, with the proviso that at least one radical R⁵, especially at least two radicals R⁵, represents a radical CH₃— CH(OR⁶)—CH₂— C(O)—, wherein the radical R⁶ comprises a radical R², as defined hereinabove.

Furthermore, according to yet another particular embodiment, in the general formula (IId″), R⁵ and R⁶, each independently of one another, identical or different, may not represent hydrogen.

Especially, according to a further particular embodiment of the method according to the invention, as a reaction product, a mixture of at least two different functionalized, especially fatty acid functionalized, preferably C₅-C₃₄-fatty acid functionalized, more preferably C₈-C₃₄-fatty acid functionalized, 3-hydroxybutyric acid polyol esters (II″′), especially 3-hydroxybutyric acid polyglycerol esters, as defined hereinabove, may be obtained, however, with the proviso that the number and position of the 3-hydroxybutyrate radicals of the general formula (II′) in the 3-hydroxybutyric acid polyol ester (II) obtained by the reduction correspond to the number and position of the 3-oxobutyrate radicals of the general formula (I′) in the 3-oxobutyric acid polyol ester (I) to be reduced.

According to another particular embodiment of the method according to the invention, as a reaction product, a mixture of at least three different functionalized, especially fatty acid functionalized, preferably C₅-C₃₄-fatty acid functionalized, more preferably C₈-C₃₄-fatty acid functionalized, 3-hydroxybutyric acid polyol esters (II″′), especially 3-hydroxybutyric acid polyglycerol esters, as defined hereinabove, may be obtained, however, with the proviso that the number and position of the 3-hydroxybutyrate radicals of the general formula (II′) in the 3-hydroxybutyric acid polyol ester (II) obtained by the reduction correspond to the number and position of the 3-oxobutyrate radicals of the general formula (I′) in the 3-oxobutyric acid polyol ester (I) to be reduced.

As previously stated, according to the method of the invention, it may be provided that in the reaction product the 3-hydroxybutyrate radical of the general formula (II′), as defined hereinabove, is in the form of the (R)-configured enantiomer.

In this context, the chirality center (asymmetric carbon atom) is located at the 3-position of the 3-hydroxybutyrate radical of the general formula (II′).

As explained hereinbefore, the method according to the invention is usually carried out in the absence of solvents and/or without any solvent (i. e. as a reaction in mass or as a reaction in substance or as a so-called bulk reaction). This has the advantage that the reaction products obtained are not contaminated with solvent and no solvent has to be removed and disposed of or recycled in a costly and energy-intensive manner after the method or reaction has been carried out. Surprisingly, the method or reaction nevertheless proceeds with high conversions and yields and at least essentially without significant by-product formation.

A preferred procedure according to the invention (including a possible upstream synthesis of the reactants used according to the invention) without subsequent functionalization of the reaction products obtained is illustrated by the following reaction or synthesis scheme (wherein, depending on the reaction procedure, either individual esters or a mixture of two or more thereof are obtained):

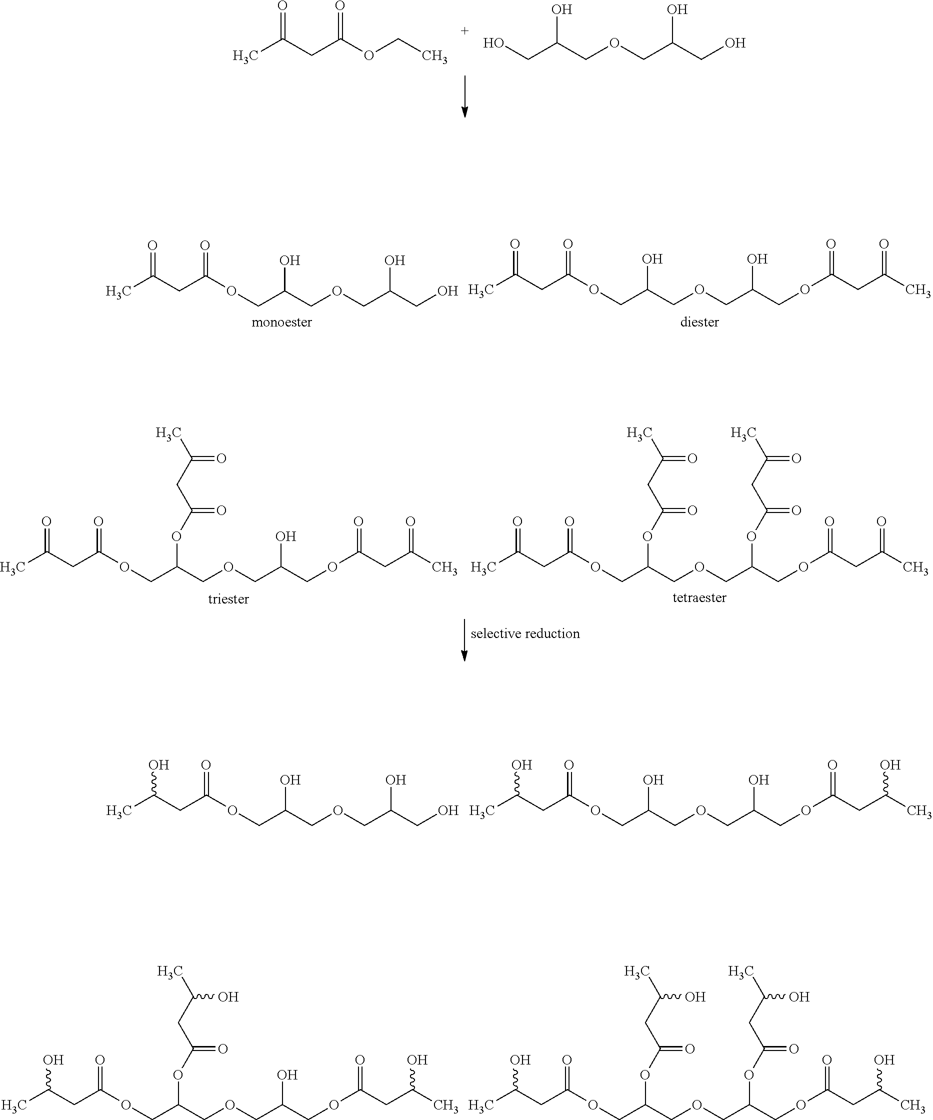

A further preferred procedure according to the invention (including a possible upstream synthesis of the starting materials used according to the invention) with subsequent functionalization of the reaction products obtained is illustrated by the following reaction or synthesis scheme (wherein, depending on the reaction procedure, either individual esters or a mixture of two or more thereof are obtained and wherein in the following reaction or synthesis scheme the radical R represents hydrogen or a linear or branched, saturated or mono- or polyunsaturated C₈-C₃₄-fatty acid radical [═(C₇-C₃₃-alkyl)-C(O) radical], however, with the proviso that at least one radical R per molecule shown does not represent H):

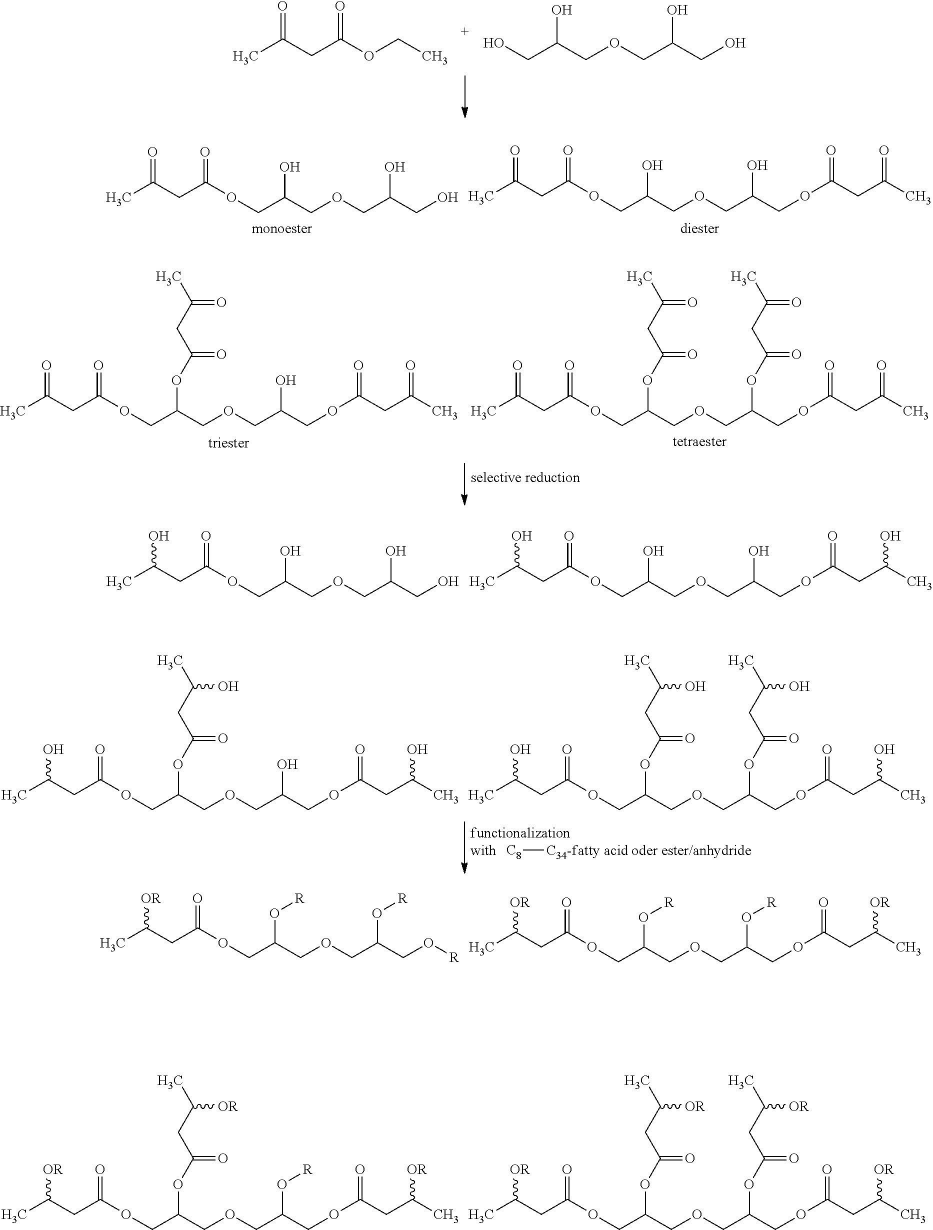

A further preferred procedure according to the invention (including a possible upstream synthesis of the starting materials used according to the invention) with chiral reduction and subsequent functionalization of the reaction products obtained ((R)-configured enantiomers) is illustrated by the following reaction or synthesis scheme (wherein the radical R represents hydrogen or a linear or branched, saturated or mono- or polyunsaturated C₈-C₃₄-fatty acid radical [═(C₇-C₃₃-alkyl)-C(O) radical], however, with the proviso that at least one radical R per molecule shown does not represent H):

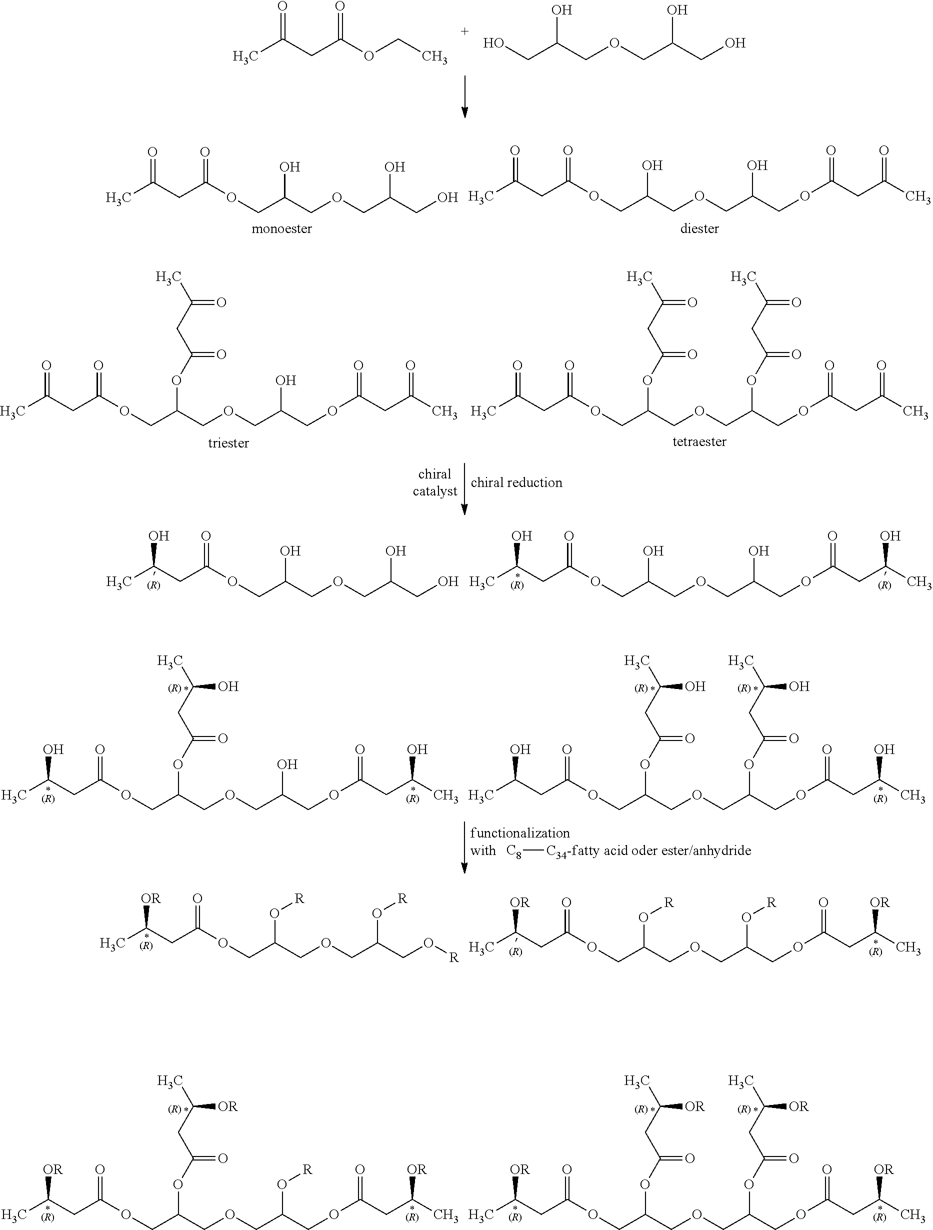

A further subject-matter—according to a second aspect of the present invention—is the reaction product obtainable according to the method according to the invention or the inventive reaction product (i. e. one or more optionally functionalized, preferentially optionally fatty acid functionalized, preferably optionally C₅-C₃₄-fatty acid functionalized, especially optionally C₈-C₃₄-fatty acid functionalized, polyol esters, especially polyglycerol esters, of 3-hydroxybutyric acid or mixtures thereof). The reaction product obtainable by the method according to the invention or the inventive reaction product is preferably enantiomerically enriched, especially enantiomerically pure, preferentially in the form of the (R)-configured enantiomer, each with reference to the chirality center (asymmetric carbon atom) of the 3-hydroxybutyrate radical of the general formula (II′) CH₃—C*H(OH)—CH₂— C(O)O—, which is generated by means of reduction and is subsequently indicated by the symbol “*”.

The object of the present invention according to this aspect of the invention is especially an optionally functionalized, preferentially optionally fatty acid functionalized, preferably optionally C₅-C₃₄-fatty acid functionalized, especially optionally C₅-C₃₄-fatty acid functionalized, 3-hydroxybutyric acid polyol ester (II), especially 3-hydroxybutyric acid polyglycerol ester, of the general formula (VIIa)

(R⁸O)ₘ—(X)—(OR⁸)ₙ (VIIa)

wherein, in the general formula (VIIa),
X represents an organic radical, especially a preferentially saturated organic radical comprising 3 to 21 carbon atoms, preferentially 4 to 21 carbon atoms, and optionally comprising 1 to 9 oxygen atoms, preferentially selected from an alkyl radical or a (poly)alkyl ether radical, especially (poly)alkylene glycol radical, more preferably selected from C₃-C₂₁-alkyl radical, preferentially C₄-C₂₁-alkyl radical, or C₃-C₂-(poly)alkyl ether radical, preferentially C₃-C₂₁-(poly)alkyl ether radical, especially C₃-C₂-(poly)alkylene glycol radical, preferentially C₄-C₂₁-(poly)alkylene glycol radical,
the variables m and n, each independently of one another, represent an integer from 1 to 10,
the radical R⁸, each independently of one another, identical or different, represents: hydrogen, a radical R², wherein the radical R² represents a radical of the type linear (straight-chained) or branched, saturated or mono- or polyunsaturated (C₁-C₃₃-alkyl)-C(O)—, especially (C₄-C₃₃-alkyl)-C(O)—, preferably (C₇-C₃₃-alkyl)-C(O)—, or a radical CH₃— C*H(OR⁶)—CH₂— C(O)—, wherein the radical R⁶ represents hydrogen or a radical R², as defined hereinabove, however, with the proviso that at least one radical R⁸, especially at least two radicals R⁸, does not represent hydrogen, and with the proviso that at least one radical R⁸, especially at least two radicals R⁸, represents a radical CH₃—C*H(OR⁶)—CH₂—C(O)—,
   wherein the optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester (II), especially 3-hydroxybutyric acid polyglycerol ester, is enantiomerically enriched, especially enantiomerically pure, preferentially in the form of the (R)-configured enantiomer, each relative to the chirality center (asymmetric carbon atom) of the 3-hydroxybutyrate radical of the general formula (II′) CH₃—C*H(OH)—CH₂— C(O)O—, which is generated by means of reduction and is subsequently indicated by the symbol “*”.

In the context of the present invention, enantiomerically enriched means the presence of at least 90%, especially at least 95%, preferentially at least 96%, preferably at least 97%, more preferably at least 98%, even more preferably at least 99%, of an enantiomer in the reaction product. Furthermore, in the context of the present invention, enantiomerically pure is to be understood as the presence of essentially 100% of an enantiomer in the reaction product.

Especially, the groups R⁸O—may be in any position of the radical X, preferentially wherein at least one group R⁸O—is terminal.

A further object of the present invention is also an optionally functionalized 3-hydroxybutyric acid polyol ester (II), especially a polyol ester as described or defined hereinabove,

wherein the optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester (II), especially 3-hydroxybutyric acid polyglycerol ester, corresponds to the general formula (VIIb)

R⁸O—CH₂—CH(OR⁸)—CH₂—[O—CH₂—CH(OR⁸)—CH₂]ₚ—OR⁸ (VIIb)

wherein, in the general formula (VIIb),
the variable p represents an integer from 0 to 6, especially from 1 to 4, preferentially 1 or 2, more preferably 1,
the radical R⁸, each independently of one another, identical or different, represents: hydrogen, a radical R², wherein the radical R² represents a radical of the type linear (straight-chained) or branched, saturated or mono- or polyunsaturated (C₁-C₃₃-alkyl)-C(O)—, especially (C₄-C₃₃-alkyl)-C(O)—, preferably (C₇-C₃₃-alkyl)-C(O)—, or a radical CH₃— C*H(OR⁶)—CH₂— C(O)—, wherein the radical R⁶ represents hydrogen or a radical R², as defined hereinabove, however, with the proviso that at least one radical R⁸, especially at least two radicals R⁸, does not represent hydrogen, and with the proviso that at least one radical R⁸, especially at least two radicals R⁸, represents a radical CH₃— C*H(OR⁶)—CH₂— C(O)—,
   wherein the optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester (II), especially 3-hydroxybutyric acid polyglycerol ester, is enantiomerically enriched, especially enantiomerically pure, preferentially in the form of the (R)-configured enantiomer, each based on the chirality center (asymmetric carbon atom) of the 3-hydroxybutyrate radical of the general formula (II′) CH₃—C*H(OH)—CH₂— C(O)O—, which is generated by means of reduction and is subsequently indicated by the symbol “*”.

Again, another object of the present invention is also an optionally functionalized 3-hydroxybutyric acid polyol ester (II), especially a polyol ester as described or defined hereinabove, wherein the optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester (II), especially 3-hydroxybutyric acid polyglycerol ester, corresponds to the general formula (VIIc)

R⁸O—CH₂—CH(OR⁸)—CH₂—O—CH₂—CH(OR⁸)—CH₂—OR⁸ (VIIc)

wherein, in the general formula (VIIc), the radical R⁸, each independently of one another, identical or different, represents: hydrogen, a radical R², wherein the radical R² represents a radical of the type linear (straight-chained) or branched, saturated or mono- or polyunsaturated (C₁-C₃₃-alkyl)-C(O)—, especially (C₄-C₃₄-alkyl)-C(O)—, preferably (C₇-C₃₃-alkyl)-C(O)—, or a radical CH₃— C*H(OR⁶)—CH₂— C(O)—, wherein the radical R⁶ represents hydrogen or a radical R², as defined hereinabove, however, with the proviso that at least one radical R⁸, especially at least two radicals R⁸, does not represent hydrogen, and with the proviso that at least one radical R⁸, especially at least two radicals R⁸, represents a radical CH₃— C*H(OR⁶)—CH₂— C(O)—,
wherein the optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester (II), especially 3-hydroxybutyric acid polyglycerol ester, is enantiomerically enriched, especially enantiomerically pure, preferentially in the form of the (R)-configured enantiomer, each based on the chirality center (asymmetric carbon atom) of the 3-hydroxybutyrate radical of the general formula (II′) CH₃— C*H(OH)—CH₂— C(O)O—, which is generated by means of reduction and is subsequently indicated by the symbol “*”.

Similarly, it is an object of the present invention to provide an optionally functionalized 3-hydroxybutyric acid polyol ester (II), especially a polyol ester as described or defined hereinabove,

wherein the optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester (II), especially 3-hydroxybutyric acid polyglycerol ester, corresponds to the general formula (VIId)

R⁸O—CH₂—CH(OR⁵)—CH₂—OR⁸ (VIId)

wherein, in the general formula (VIId), the radical R⁸, each independently of one another, identical or different, represents: hydrogen, a radical R², wherein the radical R² represents a radical of the type linear (straight-chained) or branched, saturated or mono- or polyunsaturated (C₁-C₃₃-alkyl)-C(O)—, especially (C₄-C₃₄-alkyl)-C(O)—, preferably (C₇-C₃₃-alkyl)-C(O)—, or a radical CH₃— C*H(OR⁶)—CH₂— C(O)—, wherein the radical R⁶ represents hydrogen or a radical R², as defined hereinabove, however, with the proviso that at least one radical R⁸, especially at least two radicals R⁸, does not represent hydrogen, and with the proviso that at least one radical R⁸, especially at least two radicals R⁸, represents a radical CH₃— C*H(OR⁶)—CH₂— C(O)—,
wherein the optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester (II), especially 3-hydroxybutyric acid polyglycerol ester, is enantiomerically enriched, especially enantiomerically pure, preferentially in the form of the (R)-configured enantiomer, each based on the chirality center (asymmetric carbon atom) of the 3-hydroxybutyrate radical of the general formula (II′) CH₃— C*H(OH)—CH₂— C(O)O—, which is generated by means of reduction and is subsequently indicated by the symbol “*”.

According to a particular embodiment, the object of the present invention is also a 3-hydroxybutyric acid polyol ester (II″), especially 3-hydroxybutyric acid polyglycerol ester, of the general formula (VIIa″)

(R⁹O)ₘ—(X)—(OR⁹)ₙ (VIIa″)

wherein, in the general formula (VIIa″),
X represents an organic radical, especially a preferentially saturated organic radical comprising 3 to 21 carbon atoms, preferentially 4 to 21 carbon atoms, and optionally comprising 1 to 9 oxygen atoms, preferentially selected from an alkyl radical or a (poly)alkyl ether radical, especially (poly)alkylene glycol radical, more preferably selected from C₃-C₂₁-alkyl radical, preferentially C₄-C₂₁-alkyl radical, or C₃-C₂₁-(poly)alkyl ether radical, preferentially C₄-C₂₁-(poly)alkyl ether radical, especially C₃-C₂₁-(poly)alkylene glycol radical, preferentially C₄-C₂₁-(poly)alkylene glycol radical,
the variables m and n, each independently of one another, represent an integer from 1 to 10,
the radical R⁹, each independently of one another, identical or different, represents: hydrogen or a radical CH₃— C*H(OH)—CH₂— C(O)—, however, with the proviso that at least one radical R⁹, especially at least two radicals R⁹, does not represent hydrogen,
   wherein the 3-hydroxybutyric acid polyol ester (II″), especially 3-hydroxybutyric acid polyglycerol ester, is enantiomerically enriched, especially enantiomerically pure, preferentially in the form of the (R)-configured enantiomer, each relative to the chirality center (asymmetric carbon atom) of the 3-hydroxybutyrate radical of the general formula (II′) CH₃—C*H(OH)—CH₂— C(O)O—, which is generated by means of reduction and is subsequently indicated by the symbol “*”;

Especially the groups R⁹O—may be in any position of the radical X, preferentially wherein at least one group R⁹O—is terminal.

According to another particular embodiment, the object of the present invention is also a 3-hydroxybutyric acid polyol ester (II″), especially a polyol ester as described or defined hereinabove,

wherein the 3-hydroxybutyric acid polyol ester (II″), especially 3-hydroxybutyric acid polyglycerol ester, corresponds to the general formula (VIIb″)

R⁹O—CH₂—CH(OR⁹)—CH₂—[O—CH₂—CH(OR⁹)—CH₂]ₚ—OR⁹ (VIIb″)

wherein, in the general formula (VIIb″),
the variable p represents an integer from 0 to 6, especially from 1 to 4, preferentially 1 or 2, more preferably 1,
the radical R⁹, each independently of one another, identical or different, represents: hydrogen or a radical CH₃— C*H(OH)—CH₂— C(O)—, however, with the proviso that at least one radical R⁹, especially at least two radicals R⁹, does not represent hydrogen,
   wherein the 3-hydroxybutyric acid polyol ester (II″), especially 3-hydroxybutyric acid polyglycerol ester, is enantiomerically enriched, especially enantiomerically pure, preferentially in the form of the (R)-configured enantiomer, each based on the chirality center (asymmetric carbon atom) of the 3-hydroxybutyrate radical of the general formula (II′) CH₃—C*H(OH)—CH₂— C(O)O—, which is generated by means of reduction and is subsequently indicated by the symbol “*”.

Again, another object of the present invention according to a further embodiment is also a 3-hydroxybutyric acid polyol ester (II″), especially a polyol ester as described or defined hereinabove,

wherein the 3-hydroxybutyric acid polyglycerol ester corresponds to the general formula (VIIc″)

R⁹O—CH₂—CH(OR⁹)—CH₂—O—CH₂—CH(OR⁹)—CH₂—OR⁹ (VIIc″)

wherein, in the general formula (VIIc″), the radical R⁹, each independently of one another, identical or different, represents: hydrogen or a radical CH₃— C*H(OH)—CH₂— C(O)—, however, with the proviso that at least one radical R¹, especially at least two radicals R¹, does not represent hydrogen,
wherein the 3-hydroxybutyric acid polyol ester (II″), especially 3-hydroxybutyric acid polyglycerol ester, is enantiomerically enriched, especially enantiomerically pure, preferentially in the form of the (R)-configured enantiomer, each based on the chirality center (asymmetric carbon atom) of the 3-hydroxybutyrate radical of the general formula (II′) CH₃—C*H(OH)—CH₂— C(O)O—, which is generated by means of reduction and is subsequently indicated by the symbol “*”.

A further object of this particular embodiment of the present invention is also a 3-hydroxybutyric acid polyol ester (II″), especially a polyol ester as described or defined hereinabove,

wherein the 3-hydroxybutyric acid polyol ester (II″), especially 3-hydroxybutyric acid polyglycerol ester, corresponds to the general formula (VIId″)

R⁹O—CH₂—CH(OR⁹)—CH₂—OR⁹ (VIId″)

wherein, in the general formula (VIId″), the radical R⁹, each independently of one another, identical or different, represents: hydrogen or a radical CH₃— C*H(OH)—CH₂— C(O)—, however, with the proviso that at least one radical R⁹, especially at least two radicals R⁹, does not represent hydrogen,
wherein the 3-hydroxybutyric acid polyol ester (II″), especially 3-hydroxybutyric acid polyglycerol ester, is enantiomerically enriched, especially enantiomerically pure, preferentially in the form of the (R)-configured enantiomer, each relative to the chirality center (asymmetric carbon atom) of the 3-hydroxybutyrate radical of the general formula (II′) CH₃—C*H(OH)—CH₂— C(O)O—, which is generated by means of reduction and is subsequently indicated by the symbol “*”.

According to an alternative embodiment, also an object of the present invention is a functionalized, especially fatty acid functionalized, preferably C₅-C₃₄-fatty acid functionalized, more preferably C₈-C₃₄-fatty acid functionalized, 3-hydroxybutyric acid polyol ester (II″′), especially 3-hydroxybutyric acid polyglycerol ester, of the general formula (VIIa″′)

(R⁸O)ₘ—(X)—(OR⁸) (VIIa″′)

wherein, in the general formula (VIIa″′),
X represents an organic radical, especially a preferentially saturated organic radical comprising 3 to 21 carbon atoms, preferentially 4 to 21 carbon atoms, and optionally comprising 1 to 9 oxygen atoms, preferentially selected from an alkyl radical or a (poly)alkyl ether radical, especially (poly)alkylene glycol radical, more preferably selected from C₃-C₂₁-alkyl radical, preferentially C₄-C₂₁-alkyl radical, or C₃-C₂₁-(poly)alkyl ether radical, preferentially C₄-C₂₁-(poly)alkyl ether radical, especially C₃-C₂₁-(poly)alkylene glycol radical, preferentially C₄-C₂₁-(poly)alkylene glycol radical,
the variables m and n, each independently of one another, represent an integer from 1 to 10,
the radical Ra, each independently of one another, identical or different, represents: hydrogen, a radical R², wherein the radical R² represents a radical of the type linear (straight-chained) or branched, saturated or mono- or polyunsaturated (C₁-C₃₃-alkyl)-C(O)—, especially (C₄-C₃₄-alkyl)-C(O)—, preferably (C₇-C₃₃-alkyl)-C(O)—, or a radical CH₃— C*H(OR⁶)—CH₂— C(O)—, wherein the radical R⁶ represents hydrogen or a radical R², as defined hereinabove, however, with the proviso that at least one radical R⁸, especially at least two radicals R⁸, does not represent hydrogen, and with the proviso that at least one radical R⁸, especially at least two radicals R⁸, represents a radical CH₃— C*H(OR⁶)—CH₂— C(O)—, and with the proviso that at least its radical R⁸ and/or a radical R⁶ represents a radical R²,
   wherein the functionalized, preferentially fatty acid functionalized, preferably C₅-C₃₄-fatty acid functionalized, more preferably C₈-C₃₄-fatty acid functionalized, 3-hydroxybutyric acid polyol ester (II″′), especially 3-hydroxybutyric acid polyglycerol ester, is enantiomerically enriched, especially enantiomerically pure, preferentially in the form of the (R)-configured enantiomer, each relative to the chirality center (asymmetric carbon atom) of the 3-hydroxybutyrate radical of the general formula (II′) CH₃—C*H(OH)—CH₂— C(O)O—, which is generated by means of reduction and is subsequently indicated by the symbol “*”.

Especially, the groups R⁸O—may be in any position of the radical X, preferentially wherein at least one group R⁸O—is terminal.

Especially, according to a particular embodiment, in the aforementioned general formula (VIIa″), the radical R⁸, each independently of one another, identical or different, may represent: a radical CH₃— C*H(OR⁶)—CH₂— C(O)—, wherein the radical R⁶ represents a radical R², as defined hereinabove, or else a radical R², as defined hereinabove, however, with the proviso that at least one radical R⁸, especially at least two radicals R⁸, represents a radical CH₃— C*H(OR⁶)— CH₂— C(O)—, wherein the radical R⁶ comprises a radical R², as defined hereinabove

Furthermore, according to another embodiment, in general formula (VIIa″), R⁸ and R⁶, each independently of one another, identical or different, may not represent hydrogen.

A further object according to this particular embodiment of the present invention is also a functionalized 3-hydroxybutyric acid polyol ester (II″′), especially a polyol ester as described or defined hereinabove,

wherein the functionalized, especially fatty acid functionalized, preferably C₅-C₃₄-fatty acid functionalized, more preferably C₈-C₃₄-fatty acid functionalized, 3-hydroxybutyric acid polyol ester (II″′), especially 3-hydroxybutyric acid polyglycerol ester, corresponds to the general formula (VIIb′″)

R⁸O—CH₂—CH(OR⁸)—CH₂—[O—CH₂—CH(OR⁸)—CH₂]ₚ—OR⁸ (VIIb′″)

wherein, in the general formula (VIIb″),
the variable p represents an integer from 0 to 6, especially from 1 to 4, preferentially 1 or 2, more preferably 1,
the radical R⁸, each independently of one another, identical or different, represents: hydrogen, a radical R², wherein the radical R² represents a radical of the type linear (straight-chained) or branched, saturated or mono- or polyunsaturated (C₁-C₃₄-alkyl)-C(O)—, especially (C₈-C₃₃-alkyl)-C(O)—, preferably (C₇-C₃₃-alkyl)-C(O)—, or a radical CH₃— C*H(OR⁶)—CH₂— C(O)—, wherein the radical R⁶ represents hydrogen or a radical R², as defined hereinabove, however, with the proviso that at least one radical R⁸, especially at least two radicals R⁸, does not represent hydrogen, and with the proviso that at least one radical R⁸, especially at least two radicals R⁸, represents a radical CH₃— C*H(OR⁶)—CH₂— C(O)—, and with the proviso that at least its radical Ra and/or a radical R⁶ represents a radical R²,
   wherein the functionalized, preferentially fatty acid functionalized, preferably C₅-C₃₄-fatty acid functionalized, more preferably C₅-C₃₄-fatty acid functionalized, 3-hydroxybutyric acid polyol ester (II″′), especially 3-hydroxybutyric acid polyglycerol ester, is enantiomerically enriched, especially enantiomerically pure, preferentially in the form of the (R)-configured enantiomer, each relative to the chirality center (asymmetric carbon atom) of the 3-hydroxybutyrate radical of the general formula (II′) CH₃—C*H(OH)—CH₂— C(O)O—, which is generated by means of reduction and is subsequently indicated by the symbol “*”.

Especially, according to a particular embodiment, in the general formula (VIIb″), the radical R⁸, each independently of one another, identical or different, may represent: a radical CH₃— C*H(OR⁶)—CH₂— C(O)—, wherein the radical R⁶ represents a radical R², as defined hereinabove, or else a radical R², as defined hereinabove, however, with the proviso that at least one radical R⁸, especially at least two radicals R⁸, represents a radical CH₃— C*H(OR⁶)—CH₂— C(O)—, wherein the radical R⁶ comprises a radical R², as defined hereinabove.

According to a further preferred embodiment, in general formula (VIIb″), R⁸ and R⁶ each independently, identical or different, may not represent hydrogen.

Again, another object of the present invention according to this particular embodiment is also a functionalized 3-hydroxybutyric acid polyol ester (II″′), especially a polyol ester as described or defined hereinabove,

wherein the functionalized, especially fatty acid functionalized, preferably C₅-C₃₄-fatty acid functionalized, more preferably C₈-C₃₄-fatty acid functionalized, 3-hydroxybutyric acid polyol ester (II″′), especially 3-hydroxybutyric acid polyglycerol ester, corresponds to the general formula (VIIc′″)

R⁸O—CH₂—CH(OR⁸)—CH₂—O—CH₂—CH(OR⁸)—CH₂—OR⁸ (VIIc″′)

wherein, in the general formula (VIIc″), the radical R⁸, each independently of one another, identical or different, represents: hydrogen, a radical R², wherein the radical R² represents a radical of the type linear (straight-chained) or branched, saturated or mono- or polyunsaturated (C₁-C₃₃-alkyl)-C(O)—, especially (C₄-C₃₃-alkyl)-C(O)—, preferably (C₇-C₃₄₃-alkyl)-C(O)—, or a radical CH₃— C*H(OR⁶)—CH₂— C(O)—, wherein the radical R⁶ represents hydrogen or a radical R², as defined hereinabove, however, with the proviso that at least one radical R⁸, especially at least two radicals R⁸, does not represent hydrogen, and with the proviso that at least one radical R⁸, especially at least two radicals R⁸, represents a radical CH₃— C*H(OR⁶)—CH₂— C(O)—, and with the proviso that at least its radical R⁸ and/or a radical R⁶ represents a radical R²,
wherein the functionalized, preferentially fatty acid functionalized, preferably C₅-C₃₄-fatty acid functionalized, more preferably C₈-C₃₄-fatty acid functionalized, 3-hydroxybutyric acid polyol ester (II′″), especially 3-hydroxybutyric acid polyglycerol ester, is enantiomerically enriched, especially enantiomerically pure, preferentially in the form of the (R)-configured enantiomer, each relative to the chirality center (asymmetric carbon atom) of the 3-hydroxybutyrate radical of the general formula (II′) CH₃—C*H(OH)—CH₂— C(O)O—, which is generated by means of reduction and is subsequently indicated by the symbol “*”.

According to a particular embodiment, in the general formula (VIIc″), the radical R⁸, each independently of one another, identical or different, may represent: a radical CH₃— C*H(OR⁶)—CH₂— C(O)—, wherein the radical R⁶ represents a radical R², as defined hereinabove, or else a radical R², as defined hereinabove, however, with the proviso that at least one radical R⁸, especially at least two radicals R⁸, represents a radical CH₃— C*H(OR⁶)—CH₂— C(O)—, wherein the radical R⁶ comprises a radical R², as defined hereinabove.

Especially, according to another particular embodiment, in general formula (VIIc″), R⁸ and R⁶, each independently of one another, identical or different, may not represent hydrogen.

Similarly, it is also an object of the present invention according to this particular embodiment to provide a functionalized 3-hydroxybutyric acid polyol ester (II″′), especially a polyol ester as described or defined hereinabove,

wherein the functionalized, preferentially fatty acid functionalized, preferably C₅-C₃₄-fatty acid functionalized, more preferably C₈-C₃₄-fatty acid functionalized, 3-hydroxybutyric acid polyglycerol ester corresponds to the general formula (VIId″)

R⁸O—CH₂—CH(OR⁸)—CH₂—OR⁸ (VIId″′)

wherein, in the general formula (VIId″), the radical R⁸, each independently of one another, identical or different, represents: hydrogen, a radical R², wherein the radical R² represents a radical of the type linear (straight-chained) or branched, saturated or mono- or polyunsaturated (C₁-C₃₃-alkyl)-C(O)—, especially (C₄-C₃₃-alkyl)-C(O)—, preferably (C₇-C₃₃-alkyl)-C(O)—, or a radical CH₃— C*H(OR⁶)—CH₂— C(O)—, wherein the radical R⁶ represents hydrogen or a radical R², as defined hereinabove, however, with the proviso that at least one radical R⁸, especially at least two radicals R⁸, does not represent hydrogen, and with the proviso that at least one radical R⁸, especially at least two radicals R⁸, represents a radical CH₃— C*H(OR⁶)—CH₂— C(O)—, and with the proviso that at least its radical R⁸ and/or a radical R⁶ represents a radical R²,
wherein the functionalized, preferentially fatty acid functionalized, preferably C₅-C₃₄-fatty acid functionalized, more preferably C₈-C₃₄-fatty acid functionalized, 3-hydroxybutyric acid polyol ester (II″′), especially 3-hydroxybutyric acid polyglycerol ester, is enantiomerically enriched, especially enantiomerically pure, preferentially in the form of the (R)-configured enantiomer, each relative to the center of chirality (asymmetric carbon atom) of the 3-hydroxybutyrate radical of the general formula (II′) CH₃—C*H(OH)—CH₂— C(O)O—, which is generated by means of reduction and is subsequently indicated by the symbol “*”.

Especially, according to a particular embodiment, in the aforementioned general formula (VIId″), the radical R⁸, each independently of one another, identical or different, may represent: a radical CH₃— C*H(OR⁶)—CH₂— C(O)—, wherein the radical R⁶ represents a radical R², as defined hereinabove, or else a radical R², as defined hereinabove, however, with the proviso that at least one radical Ra, especially at least two radicals Ra, represents a radical CH₃— C*H(OR⁶)—CH₂— C(O)—, wherein the radical R⁶ comprises a radical R², as defined hereinabove.

Furthermore, according to another particular embodiment, in the general formula (VIId″), the radicals R⁸ and R⁶, each independently of one another, identical or different, may not represent hydrogen.

A further object of the present invention according to this aspect of the invention, in accordance with a particular embodiment, is a mixture comprising at least two, preferentially at least three, different optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol esters (II), especially 3-hydroxybutyric acid polyglycerol esters, as defined hereinabove.

The reaction product obtainable according to the inventive method or the inventive reaction product as defined hereinabove, respectively, and/or the optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester (II), especially 3-hydroxybutyric acid polyglycerol ester, obtainable according to the inventive production method or the inventive optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester (II), especially 3-hydroxybutyric acid polyglycerol ester, as defined hereinabove, respectively, and/or the mixture, obtainable according to the inventive production method or the inventive mixture as defined hereinabove, respectively, comprises a multitude of advantages and special features compared to the prior art:

As the applicant has surprisingly found out, the reaction product obtainable according to the inventive method or the inventive reaction product as defined hereinabove, respectively, and/or the optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester (II), especially 3-hydroxybutyric acid polyglycerol ester, as defined hereinabove, respectively, and/or the mixture, obtainable according to the inventive production method or the inventive mixture as defined hereinabove, respectively, is suitable as a precursor or metabolite of 3-hydroxybutyric acid or its salts, since, on the one hand, it is converted physiologically, especially in the gastrointestinal tract, to 3-hydroxybutyric acid or its salts and, on the other hand, it simultaneously comprises a good physiological compatibility or tolerability, especially with regard to non-toxicity and acceptable organoleptic properties. Especially, the sustained release of the physiologically active substance in the gastrointestinal tract is advantageous in the medical field, since in this way the active substance 3-hydroxybutyric acid can be made available over a longer period of time, thus enabling sustained release keto therapy.

Moreover, the reaction product obtainable according to the inventive method or the inventive reaction product as defined hereinabove, respectively, and/or the optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester (II), especially 3-hydroxybutyric acid polyglycerol ester, as defined hereinabove, respectively, and/or the mixture, obtainable according to the inventive production method or the inventive mixture as defined hereinabove, respectively, is easily accessible or available on a large scale on a synthetic basis, even on a commercial scale, and with the required pharmaceutical or pharmacological quality.

Additionally, the reaction product obtainable according to the inventive method or the inventive reaction product as defined hereinabove, respectively, and/or the optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester (II), especially 3-hydroxybutyric acid polyglycerol ester as defined hereinabove, respectively, and/or the mixture, obtainable according to the inventive production method or the inventive mixture as defined hereinabove, respectively, can, if necessary, be provided in enantiomerically pure or enantiomerically enriched form.

The reaction product obtainable according to the inventive method or the inventive reaction product as defined hereinabove, respectively, and/or the optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester (II), especially 3-hydroxybutyric acid polyglycerol ester, as defined hereinabove, respectively, and/or the mixture, obtainable according to the inventive production method or the inventive mixture as defined hereinabove, respectively, thus represents an efficient pharmacological drug target in the context of keto-body therapy of the human or animal body.

In the following, the remaining aspects of the invention are explained in more detail.

A further subject-matter of the present invention—according to a third aspect of the present invention—is a pharmaceutical composition, especially a drug or medicament, which comprises a reaction product obtainable according to the inventive production method or the inventive reaction product as defined hereinabove, respectively, and/or one or more optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester, as defined hereinabove, respectively.

Especially, according to this aspect of the invention, the present invention relates to a pharmaceutical composition for the prophylactic and/or therapeutic treatment or for use in the prophylactic and/or therapeutic treatment of diseases of the human or animal body. This may especially concern diseases associated with a disorder of the energy metabolism, especially keto-body metabolism, such as especially craniocerebral trauma, stroke, hypoxia, cardiovascular diseases such as myocardial infarction, refeeding syndrome, anorexia, epilepsy, neurodegenerative diseases such as dementia, Alzheimer's disease, Parkinson's disease, multiple sclerosis and amyotrophic lateral sclerosis, fat metabolic diseases such as glucose transporter defect (GLUT1 defect), VL-FAOD and mitochondriopathies such as mitochondrial thiolase defect, Huntington's disease, cancers such as T-cell lymphomas, astrocytomas and glioblastomas, HIV, rheumatic diseases such as rheumatoid arthritis and arthritis urica, diseases of the gastrointestinal tract such as chronic inflammatory bowel diseases, especially ulcerative colitis and Crohn's disease, lyosomal storage diseases such as sphingolipidosis, especially Niemann-Pick disease, diabetes mellitus and effects or side-effects of chemotherapy.

Again, a further subject-matter of the present invention—according to a fourth aspect of the present invention—is a reaction product obtainable according to the inventive production method or the inventive reaction product as defined hereinabove, respectively, and/or one or more optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester, as defined hereinabove, respectively, for the prophylactic and/or therapeutic treatment or for use in the prophylactic and/or therapeutic treatment of diseases of the human or animal body, especially diseases associated with a disorder of the energy metabolism, especially keto-body metabolism, such as especially craniocerebral trauma, stroke, hypoxia, cardiovascular diseases such as myocardial infarction, refeeding syndrome, anorexia, epilepsy, neurodegenerative diseases such as dementia, Alzheimer's disease, Parkinson's disease, multiple sclerosis and amyotrophic lateral sclerosis, fat metabolic diseases such as glucose transporter defect (GLUT1 defect), VL-FAOD and mitochondriopathies such as mitochondrial thiolase defect, Huntington's disease, cancers such as T-cell lymphomas, astrocytomas and glioblastomas, HIV, rheumatic diseases such as rheumatoid arthritis and arthritis urica, diseases of the gastrointestinal tract such as chronic inflammatory bowel diseases, especially ulcerative colitis and Crohn's disease, lyosomal storage diseases such as sphingolipidosis, especially Niemann-Pick disease, diabetes mellitus and effects or side-effects of chemotherapy.

Likewise, a further subject-matter of the present invention—according to a fifth aspect of the present invention—is the use of a reaction product obtainable according to the inventive production method or the inventive reaction product as defined hereinabove, respectively, and/or one or more optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester, as defined hereinabove, respectively, for the prophylactic and/or therapeutic treatment or for producing a pharmaceutical for the prophylactic and/or therapeutic treatment of diseases of the human or animal body, especially diseases associated with a disorder of the energy metabolism, especially keto-body metabolism, such as especially craniocerebral trauma, stroke, hypoxia, cardiovascular diseases such as myocardial infarction, refeeding syndrome, anorexia, epilepsy, neurodegenerative diseases such as dementia, Alzheimer's disease, Parkinson's disease, multiple sclerosis and amyotrophic lateral sclerosis, fat metabolic diseases such as glucose transporter defect (GLUT1 defect), VL-FAOD and mitochondriopathies such as mitochondrial thiolase defect, Huntington's disease, cancers such as T-cell lymphomas, astrocytomas and glioblastomas, HIV, rheumatic diseases such as rheumatoid arthritis and arthritis urica, diseases of the gastrointestinal tract such as chronic inflammatory bowel diseases, especially ulcerative colitis and Crohn's disease, lyosomal storage diseases such as sphingolipidosis, especially Niemann-Pick disease, diabetes mellitus and effects or side-effects of chemotherapy.

Likewise, a further subject-matter of the present invention—according to a sixth aspect of the present invention—is the use of a reaction product obtainable according to the inventive production method or the inventive reaction product as defined hereinabove, respectively, and/or one or more optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester, as defined hereinabove, respectively, for the prophylactic and/or therapeutic treatment or for producing a medicament for the prophylactic and/or therapeutic treatment of or for the application for catabolic metabolic states, such as hunger, diets or low-carbohydrate nutrition.

Likewise, a further subject-matter of the present invention—according to a seventh aspect of the present invention—is a food and/or a food product, which comprises a reaction product obtainable according to the inventive production method or the inventive reaction product as defined hereinabove, respectively, and/or one or more optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester, as defined hereinabove, respectively.

According to a particular embodiment, the food and/or the food product may essentially be a dietary supplement, a functional food, a novel food, a food additive, a food supplement, a dietary food, a power snack, an appetite suppressant or a strength and/or endurance sport supplement.

Finally, yet another subject-matter of the present invention—according to an eighth aspect of the present invention—is the use of a reaction product, as defined hereinabove, obtainable according to the inventive production method and/or one or more optionally functionalized, preferentially optionally fatty acid functionalized, preferably optionally C₅-C₃₄-fatty acid functionalized, especially optionally C₈-C₃₄-fatty acid functionalized, 3-hydroxybutyric acid polyol ester, as defined hereinabove, in a food and/or a food product.

According to this aspect of the invention, the food and/or the food product may especially be a dietary supplement, a functional food, a novel food, a food additive, a food supplement, a dietary food, a power snack, an appetite suppressant or a strength and/or endurance sports supplement.

Further embodiments, modifications and variations of the present invention are readily recognizable or realizable by a person skilled in the art when reading the description, without leaving the scope of the present invention.

The present invention is illustrated by the following examples, which are not intended to limit the present invention in any way, but only to explain the exemplary and non-limiting implementation and configuration of the present invention.

### EXAMPLES OF EMBODIMENTS

### Abbreviations Used

BHB=3-BHB=3-hydroxybutyric acid radical or 3-hydroxybutyrate radical
3-BHB-FS=3-hydroxybutyric acid (i. e. free acid)
PG(2)=diglycerol: HO—CH₂— CH(OH)—CH₂— O—CH₂— CH(OH)—CH₂— OH
PG(3)=polyglycerol: HO—CH₂— CH(OH)—CH₂— [O—CH₂— CH(OH)—CH₂]₂— OH
DCM=Dichloromethane: CH₂Cl₂
Pd/C=palladium on activated carbon
Raney nickel=fine grains of a nickel/aluminum alloy
Wilkinson catalyst=homogeneous catalyst with the formula Rh(PPh₃)₃Cl

### Examples of Production

The inventive production method is illustrated by the following examples. The relevant general reaction scheme is shown and explained in the general description section.

### Example 1

### Production of 3-Hydroxybutyric Acid Diglycerol Ester Mixtures from 3-Oxobutyric Acid Diglycerol Ester Mixtures by Chiral Reduction

In a 250-ml-multi-neck flask, 100 ml of an isopropanol/water mixture (9:1) and 5 g of a 3-oxobutyric acid diglycerol ester mixture (i. e. mono-, di-, tri- and tetraglycerides of 3-oxobutyric acid with diglycerol as polyol) and 1 g Chiralidon® R (chiral catalyst for enantioselective synthesis) are provided.

The reaction mixture is stirred at room temperature for eight days. The enzyme is then filtered off and the reaction product is dried. A corresponding mixture of (R)-3-hydroxybutyric acid diglycerol esters (i. e. mono-, di-, tri- and tetraglycerides of 3-(R)-hydroxybutyric acid with diglycerol) and minor amounts of unreacted 3-oxobutyric acid diglycerol esters are obtained, which are separated by standard methods (chromatographic or distillative). Characterization is performed by GC, GPC and GC-MS.

Part of the obtained mixture of diglycerol esters of 3-hydroxybutyric acid is chromatographically separated into the individual diglycerol esters (i. e. mono-diglycerol ester, di-diglycerol ester, tri-diglycerol ester, etc.), and the individual diglycerol esters are each obtained as pure substances.

### Example 2

### Production of 3-Hydroxybutyric Acid Diglycerol Ester Mixtures from 3-Oxobutyric Acid Diglycerol Ester Mixtures by Means of Non-Chiral Reduction

In a 300-ml-pressure autoclave, 50 g of a 3-oxobutyric acid diglycerol ester mixture is dissolved in 100 ml methanol and 1 to 5% by weight Pd/C is added and the mixture is stirred at 50 to 100° C. at 20 to 50 bar hydrogen pressure for 8 to 24 h. The methanol is then filtered off. After filtering off the Pd/C, the methanol is distilled off. A racemic mixture of (S)/(R)-3-hydroxybutyric acid diglycerol esters and small amounts of unreacted 3-oxobutyric acid diglycerol esters are obtained, which are separated by standard methods (chromatographic or distillative). Characterization is performed by GC, GPC and GC-MS.

Part of the obtained mixture of diglycerol esters of 3-hydroxybutyric acid is chromatographically separated into the individual diglycerol esters (i. e. mono-diglycerol esters, di-diglycerol esters, tri-diglycerol esters, etc.) and the individual diglycerol esters are each obtained as pure substances.

### Example 3

### Production of 3-Hydroxybutyric Acid Diglycerol Ester Mixtures from 3-Oxobutyric Acid Diglycerol Ester Mixtures by Means of Non-Chiral Reduction

In a 100-ml-multi-neck flask, 15 g of a 3-oxobutyric acid-diglycerol ester mixture is provided in 50 ml ethanol.

The reaction mixture is cooled to 10° C. and 1 g sodium borohydride (NaBH₄) is added stepwise. After 30 min, the reaction mixture is neutralized by adding dilute hydrochloric acid. The solvent is then removed and the radical is taken up with aqueous potassium hydrogen phosphate solution (K₂HPO₄) and extracted with isopropanol. The solvent is then distilled off and the radical is suspended in DCM. After filtration and once more removal of the solvent, a racemic mixture of (S)/(R)-3-hydroxybutyric acid diglycerol esters with minor residual amounts of 3-oxobutyric acid diglycerol monoester is obtained. The unreacted 3-oxobutyric acid diglycerol monoester is distilled off under vacuum. The reaction products are characterized by GC, GPC and GC-MS.

Part of the obtained mixture of diglycerol esters of 3-hydroxybutyric acid is chromatographically separated into the individual diglycerol esters (i. e. mono-diglycerol esters, di-diglycerol esters, tri-diglycerol esters, etc.) and the individual diglycerol esters are each obtained as pure substances.

### Further Production of 3-Hydroxybutyric Acid Polyol Ester Mixtures

The preceding experiments are each repeated, however, with other 3-oxobutyric acid polyol esters (namely with glycerol, polyglycerol PG(3) and 1,2-pentanediol as polyol components). Comparable results are obtained. Purification or fractionation are carried out in the same way.

### Functionalization Experiments

The mono-/di-/tri-/tetra-diglycerol ester mixtures obtained in the preceding experiments and the individual diglycerol esters in pure form separated chromatographically therefrom are each subsequently functionalized by reaction with docosahexaenoic acid anhydride in order to carry out esterification with docosahexaenoic acid on any remaining (i. e. still free) OH-groups of the 3-BHB radical and/or the polyol radical. The general reaction scheme for this is shown and explained in the general description section.

The experiments show that the intended functionalization by reaction with docosahexaenoic acid anhydride leads to the desired functionalized (i. e. fatty acid functionalized or fatty acid esterified) products (i. e. esterification of free OH-groups of the 3-BHB radical and/or the polyol radical with docosahexaenoic acid), as confirmed by corresponding analytics.

Comparable functionalization experiments are also carried out with eicosapentaenoic acid anhydride on the one hand and with a mixture of docosahexaenoic acid anhydride/eicosapentaenoic acid anhydride on the other hand and lead to analogous results (i. e. esterification of free OH-groups of the 3-BHB radical and/or the polyol radical in each case), as confirmed by corresponding analysis. The experiments show that the intended functionalization also leads to the desired functionalized (i. e. fatty acid functionalized or fatty acid esterified) products (i. e. esterification of free OH-groups of the 3-BHB radical and/or the polyol radical) by reaction with eicosapentaenoic acid anhydride as well as with the mixture of docosahexaenoic acid anhydride/eicosapentaenoic acid anhydride, as confirmed by corresponding analytics.

### Further Functionalization Experiments

The mono-/di-/tri-/tetra-diglycerol ester mixtures obtained in the preceding experiments and the individual diglycerol esters in pure form separated chromatographically therefrom are each subsequently functionalized by reaction with docosahexaenoic acid to obtain the fatty acid functionalized or fatty acid esterified products esterified at free OH-groups of the 3-BHB radical and/or the polyol radical.

The experiments show that the intended functionalization by reaction with docosahexaenoic acid leads to the desired products (i. e. esterification of free OH-groups of the 3-BHB radical and/or the polyol radical), as confirmed by corresponding analysis.

Comparable functionalization experiments are also carried out in each case with eicosapentaenoic acid and oleic acid and lead to analogous results (i. e. esterification of free OH-groups of the 3-BHB radical and/or the polyol radical), as confirmed by corresponding analysis. The experiments show that the intended functionalization also leads to the desired products by reacting each with eicosapentaenoic acid and oleic acid (i. e. esterification of the free OH-groups), as confirmed by corresponding analytics.

### Further Production of Non-Functionalized and Functionalized 3-BHB Polyol Ester Mixtures

a) All preceding experiments are each repeated, however, with different 3-oxobutyric acid polyol esters (namely in each case with 3-oxobutyric acid polyglycerol ester PG(3) and with 3-oxobutyric acid 1,2-pentanediol ester). The aforementioned polyol esters 3-oxobutyric acid polyglycerol ester PG(3) and 3-oxobutyric acid 1,2-pentanediol esters are efficiently converted to the desired products (i. e. the corresponding 3-hydroxybutyric acid polyol esters) both catalytically (chiral and nonchiral) and autocatalytically. Comparable results to the previous experiments are obtained. Purification and separation or fractionation and analysis are performed in the same manner. Subsequently, functionalizations are carried out in the previously described manner, which lead to the corresponding results.
b) The experiments with 3-oxobutyric acid polyol esters each with 1,2-pentanediol or diglycerol or polyglycerol PG(3) as polyol are repeated with Raney nickel as a catalyst and hydrogen as reducing agent at temperatures between 75 and 110° C. Comparable results are obtained. Purification, separation or fractionation and analysis are carried out in the same way. Subsequently, functionalizations are carried out in the previously described manner, which lead to the corresponding results.
c) The experiments with 3-oxobutyric acid polyol esters using 1,2-pentanediol, diglycerol and polyglycerol PG(3) as polyol are repeated with the Wilkinson catalyst (Rh(PPh₃)₃Cl) as a catalyst and hydrogen as reducing agent at temperatures between 75 and 110° C. Comparable results are obtained. Purification, separation or fractionation and analysis are carried out in the same way. Subsequently, functionalizations are carried out in the previously described manner, which lead to the corresponding results.
d) Since especially the 3-BHB diglycerol esters only have a slightly bitter taste, especially these esters are an efficient product group for a therapeutic application. Therefore, the reduction of a 3-oxobutyric acid diglycerol ester mixture is carried out on a larger scale (2 to 4 kg).
   First, the stoichiometric reaction conditions of the previous experiment are applied in a 2 kg scale using Chiralidon® R as a catalyst and isopropanol as reducing agent. After stirring at room temperature for 10 days, a fully reduced reaction product (i. e. 3-hydroxybutyric acid diglycerol ester mixture) is obtained. This is followed by separation of the enzyme and purification by distillation.
   Half of this reaction product (approx. 1 kg) is completely functionalized with docosahexaenoic acid anhydride (200 mol-% excess). Analysis confirm that no free hydroxyl groups remain in the functionalized product. After distilling off the excess fatty acid anhydride, a respective fully functionalized mixture of 3-hydroxybutyric acid diglycerol esters is obtained.
   The fully functionalized mixture of diglycerol esters of 3-hydroxybutyric acid only has a slightly bitter taste and is organoleptically acceptable and compatible.

### Production of 3-Oxobutyric Acid Diglycerol Esters (=Reactant)

25 g 3-oxobutyric acid ethyl ester (ethyl acetoacetate or acetoacetic ester) and 6.5 g diglycerol are provided in a 100-ml-multi-neck flask equipped with a dephlegmator (partial condenser) and distillation bridge.

At a temperature of 50° C., 0.35 g immobilized enzyme (CALB lipase on polymer support, derived from Candida antarctica, e. g. Novozym® 435) is added. The reaction mixture is stirred at 50° C. for 24 h. The enzyme is then filtered off and the excess 3-oxobutyric acid ethyl ester is distilled off under vacuum.

The reaction product obtained is a 3-oxobutyric acid diglycerol ester and, after analytical analysis, consists of the following composition: 3-oxobutyric acid mono-diglycerol ester 45%, 3-oxobutyric acid di-diglycerol ester 48%, 3-oxobutyric acid tri-diglycerol ester 7%. Characterization is performed by GC, GPC and GC-MS.

During purification, reactants and reaction by-products are removed so that a pure mixture is obtained. Part of the mixture is subjected to separation by chromatography so that the various diglycerol esters are each obtained as pure substances (i. e. pure 3-oxobutyric acid mono-diglycerol ester, pure 3-oxobutyric acid di-diglycerol ester and pure 3-oxobutyric acid tri-diglycerol ester). Another part of the mixture is subjected to fractional distillation separation.

### Further Production of 3-Oxobutyric Acid Diglycerol Esters (=Reactant)

106 g ethyl 3-oxobutyric acid ester (ethyl acetoacetate or acetoacetic acid ester) and 29 g diglycerol are provided in a 250-ml-multi-neck flask equipped with a dephlegmator (partial condenser) and distillation bridge.

At a temperature of 100° C., 1.4 g 30% methanolic NaOMe solution is added under stirring. The ethanol formed during the reaction is continuously distilled off. After a reaction time of 5 h, the reaction mixture is cooled and washed with NaCl solution. The crude ester mixture is then dried and the excess ethyl 3-oxobutyric acid ester is distilled off under vacuum.

The reaction product is a 3-oxobutyric acid diglycerol ester with the following composition: 3-oxobutyric acid mono-diglycerol ester 26%, 3-oxobutyric acid di-diglycerol ester 51%, 3-oxobutyric acid tri-diglycerol ester 22%, 3-oxobutyric acid tetra-diglycerol ester 1%. Characterization is carried out by GC, GPC and GC-MS.

During purification, reactants and reaction by-products are removed so that a pure mixture is obtained. Part of the mixture is subjected to separation by chromatography so that the various diglycerol esters are each obtained as pure substances (i. e. pure 3-oxobutyric acid mono-diglycerol ester, pure 3-oxobutyric acid di-diglycerol ester, pure 3-oxobutyric acid tri-diglycerol ester, etc.). Another part of the mixture is subjected to separation by fractional distillation.

### Physiological Application Tests: In-Vitro Digestion Tests

### Digestion Experiments (Splitting or Cleavage Experiments) of Inventive

### 3-BHB Diglycerol Ester Mixtures

By means of cleavage experiments it is shown that 3-BHB diglycerol esters produced according to the invention or their mixtures as well as their functionalized derivatives/analogues, can be cleaved in the human gastrointestinal tract.

The starting mixture used is, on the one hand, a purified enantiomerically pure mixture obtained by the method of the invention each comprising (R)-configured 3-hydroxybutyric acid mono-diglycerol ester, 3-hydroxybutyric acid di-diglycerol ester, 3-hydroxybutyric acid tri-diglycerol ester and 3-hydroxybutyric acid tetra-diglycerol ester and, on the other hand, a purified, enantiomerically pure fatty acid-functionalized mixture of (R)-configured 3-hydroxybutyric acid mono-diglycerol ester, 3-hydroxybutyric acid di-diglycerol ester, 3-hydroxybutyric acid tri-diglycerol ester and 3-hydroxybutyric acid tetra-diglycerol ester obtained by the method of the invention.

For the cleavage experiments under near-body conditions two media are investigated:
FaSSGF, which simulates the stomach
FaSSIF, which simulates the intestinal tract

Both media are from the company Biorelevant®, Ltd. in Great Britain. In addition, in some experiments porcine pancreas is added (Panzytrat® 40,000, Fa. Allergan).

The results of the cleavage experiments in a FaSSGF or FaSSIF medium with Panzytrat® and without Panzytrat® (both 35° C., 24 h) show that the samples hydrolyze under FaSSGF conditions with Panzytrat® and without Panzytrat®; this is mainly due to the low pH value (pH=1.6) of the medium. Under FaSSIF conditions, a lower conversion using Panzytrat® takes place.

In all experiments, it can be seen that the cascade (tetraester becomes triester, triester becomes diester, etc.) continues until the desired free acid 3-hydroxybutyric acid (3-BHB-FS) is obtained.

### Further Digestion Experiments (Cleavage Experiments) of Inventive

### 3-BHB Diglycerol Ester Mixtures

Cleavage Experiments with Pancreatin

2 g of an enantiomerically pure mixture produced as described above, based on each (R)-configured 3-hydroxybutyric acid mono-diglycerol ester, 3-hydroxybutyric acid di-diglycerol ester, 3-hydroxybutyric acid tri-diglycerol ester and 3-hydroxybutyric acid tetra-diglycerol ester, and a corresponding fatty acid functionalized mixture are each dissolved in 50 g water and 0.5 g (1% by weight) pancreatin is added. The pancreatin is used in the form of the commercially available product Panzytrat® 40,000 from the Allergan company. The whole mixture is stirred on a hot plate at 50° C.; the course of the reaction is determined and monitored by continuously recording the acid number over time. The acid number increases in each case over the observation period (cleavage of the fatty acid functionalized 3-hydroxybutyric acid-diglycerol ester mixture to the free acid). The conversion/time course of the aqueous cleavage of the mixture of esters according to the invention by means of pancreatin, including the increase in the acid number over time, demonstrates the desired decomposition of the educt mixture to the free acid. This is confirmed by appropriate analysis. The experiment proves that the starting mixture (educt mixture) according to the invention is a suitable physiological precursor for 3-hydroxybutyric acid for the corresponding keto-body therapies.

The test is repeated and verified on the basis of the individual esters in pure form. Comparable results are obtained, i. e. both the 3-hydroxybutyric acid mono-diglycerol ester and the 3-hydroxybutyric acid di-diglycerol ester as well as the 3-hydroxybutyric acid tri-diglycerol ester and the 3-hydroxybutyric acid tetra-diglycerol ester as well as their functionalized derivatives are cleaved by pancreatin to the free 3-hydroxybutyric acid (3-BHB-FS).

The previously described cleavage experiments prove that the preferably enantiomerically pure and optionally functionalized, preferentially optionally fatty acid functionalized, preferably optionally C₅-C₃₄-fatty acid functionalized, especially optionally C₈-C₃₄-fatty acid functionalized, polyol esters, especially polyglycerol esters, of 3-hydroxybutyric acid are efficient precursors or metabolites of free 3-hydroxybutyric acid or its salts, especially with regard to their intended effect, which are present in physiologically tolerable or physiologically compatible form.

1-82. (canceled)

83. A method for producing a polyglycerol ester of 3-hydroxybutyric acid which polyglycerol ester is optionally C₅-C₃₄-fatty acid functionalized,
wherein an optionally C₅-C₃₄-fatty acid functionalized 3-oxobutyric acid polyglycerol ester (I) which comprises at least one 3-oxobutyrate radical of general formula (I′)
CH₃—C(O)—CH₂—C(O)O— (I′)
is subjected, by means of at least one reducing agent, to a selective reduction of the at least one 3-oxobutyrate radical of the general formula (I′) positioned at the keto group —C(O)— of the acetyl function CH₃—C(O)—, so as to result a 3-hydroxybutyrate radical of general formula (II′)
CH₃—CH(OH)—CH₂—C(O)O— (II′);
wherein the optionally C₅-C₃₄-fatty acid functionalized polyglycerol ester (I) used as a starting compound corresponds to general formula (Ib)
R¹O—CH₂—CH(OR¹)—CH₂—[O—CH₂—CH(OR¹)—CH₂]ₚ—OR¹ (Ib)
wherein, in the general formula (Ib),
the variable p represents an integer from 1 to 4,
the radical R¹, each independently of one another, identical or different, represents: hydrogen, CH₃—C(O)—CH₂—C(O)—or a radical R², wherein the radical R² represents a radical of the type linear or branched, saturated or mono- or polyunsaturated (C₄-C₃₃-alkyl)-C(O)—, however, with the proviso that at least one radical R¹ does not represent hydrogen and with the proviso that at least one radical R¹ represents a radical CH₃— C(O)—CH₂—C(O)—; and
wherein the reducing agent is selected from the group of hydrogen, hydrides and alcohols as well as mixtures thereof; and
wherein the reduction is carried out as a complete reduction or else as a non-complete reduction of all keto groups —C(O)— of the acetyl function CH₃—C(O)— of the at least one 3-oxobutyrate radical of the general formula (I′) to a 3-hydroxybutyrate radical of the general formula (II′);
so that, as a reaction product, an optionally C₅-C₃₄-fatty acid functionalized 3-hydroxybutyric acid polyglycerol ester (II) is obtained.

84. The method according to claim 83,
wherein the reducing agent is selected from the group consisting of hydrogen, inorganic hydrides and C₁-C₃₄-alcohols as well as mixtures thereof.

85. The method according to claim 83,
wherein the reduction is carried out autocatalytically or in the presence of a catalyst.

86. The method according to claim 83,
wherein the reduction is carried out in the presence of a catalyst;
wherein the catalyst is recycled after the reduction has been carried out; and
wherein the catalyst is selected from the group consisting of enzymes, metals and metal compounds.

87. The method according to claim 83,
wherein the reducing agent is used in molar amounts, based on to the keto groups —C(O)— of the acetyl function CH₃—C(O)— of the at least one 3-oxobutyrate radical of the general formula (I′), in a range of from equimolar amount up to a molar excess of 200 mol-%.

88. The method according to claim 83,
wherein the reduction is carried out such that the reaction product comprises at least one acetyl function CH₃—C(O)—.

89. The method according to claim 83,
wherein the method furthermore comprises at least one of steps (i) and (ii):
(i) hydroxyl groups still present in the reaction product (II) after the reduction has been carried out are at least partially functionalized via esterification,
(ii) ester groups present in the reaction product (II) after the reduction has been carried out are partially transesterified;
wherein esterification and transesterification are carried out by means of a C₅-C₃₄-fatty acid in its free form or in the form of its ester or anhydride.

90. A reaction product in the form of an optionally C₅-C₃₄-fatty acid functionalized 3-hydroxybutyric acid polyglycerol ester (II) as obtainable according to the method of claim 83.

91. An optionally C₅-C₃₄-fatty acid functionalized 3-hydroxybutyric acid polyglycerol ester of general formula (IIb)
R⁵O—CH₂—CH(OR⁵)—CH₂—[O—CH₂—CH(OR⁵)—CH₂]ₚ—OR⁵ (IIb)
wherein, in the general formula (IIb),
the variable p represents an integer from 1 to 4,
the radical R⁵, each independently of one another, identical or different,
represents: hydrogen, a radical R², wherein the radical R² represents a radical of the type linear or branched, saturated or mono- or polyunsaturated (C₄-C₃₃-alkyl)-C(O)—, wherein the radical R⁶ represents hydrogen or a radical R² as defined hereinabove, however, with the proviso that at least one radical R⁵ does not represent hydrogen, and with the proviso that at least one radical R⁵ represents a radical CH₃— CH(OR⁶)—CH₂—C(O)—.

92. The optionally C₅-C₃₄-fatty acid functionalized 3-hydroxybutyric acid polyglycerol ester (II) according to claim 91,
wherein the optionally C₅-C₃₄-fatty acid functionalized 3-hydroxybutyric acid polyglycerol ester (II) is a 3-hydroxybutyric acid polyglycerol ester of the general formula (IIb″)
R⁷O—CH₂—CH(OR⁷)—CH₂—[O—CH₂—CH(OR⁷)—CH₂]ₚ—OR⁷ (IIb″)
wherein, in the general formula (IIb″),
the variable p represents an integer from 1 to 4,
the radical R⁷, each independently of one another, identical or different, represents hydrogen or CH₃—CH(OH)—CH₂—C(O)—, however, with the proviso that at least one radical R⁷ does not represent hydrogen.

93. The optionally C₅-C₃₄-fatty acid functionalized 3-hydroxybutyric acid polyglycerol ester (II) according to claim 91,
wherein the optionally C₅-C₃₄-fatty acid functionalized 3-hydroxybutyric acid polyglycerol ester (II) is a C₅-C₃₄-fatty acid functionalized 3-hydroxybutyric acid polyglycerol ester of general formula (IIb″′)
R⁵O—CH₂—CH(OR⁵)—CH₂—[O—CH₂—CH(OR⁵)—CH₂]ₚ—OR⁵ (IIb″′)
wherein, in the general formula (IIb″),
the variable p represents an integer from 1 to 4,
the radical R⁵, each independently of one another, identical or different, represents: hydrogen, a radical R², wherein the radical R² represents a radical of the type linear or branched, saturated or mono- or polyunsaturated (C₄-C₃₃-alkyl)-C(O)— or a radical CH₃— CH(OR⁶)—CH₂—C(O)—, wherein the radical R⁶ represents hydrogen or a radical R² as defined hereinabove, however, with the proviso that at least one radical R⁵ does not represent hydrogen, and with the proviso that at least one radical R⁵ represents a radical CH₃— CH(OR⁶)—CH₂—C(O)—, and with the proviso that at least one of radicals R⁵ and R⁶ represents a radical R².

94. A mixture comprising at least two different optionally C₅-C₃₄-fatty acid functionalized 3-hydroxybutyric acid polyglycerol esters (II) according to claim 91.

95. A pharmaceutical composition comprising at least one optionally C₅-C₃₄-fatty acid functionalized 3-hydroxybutyric acid polyglycerol ester of general formula (IIb)
R⁵O—CH₂—CH(OR⁵)—CH₂—[O—CH₂—CH(OR⁵)—CH₂]ₚ—OR⁵ (IIb)
wherein, in the general formula (IIb),
the variable p represents an integer from 1 to 4,
the radical R⁵, each independently of one another, identical or different, represents: hydrogen, a radical R², wherein the radical R² represents a radical of the type linear or branched, saturated or mono- or polyunsaturated (C₄-C₃₃-alkyl)-C(O)—, wherein the radical R⁶ represents hydrogen or a radical R² as defined hereinabove, however, with the proviso that at least one radical R⁵ does not represent hydrogen, and with the proviso that at least one radical R⁵ represents a radical CH₃— CH(OR⁶)—CH₂—C(O)—.

96. The pharmaceutical composition according to claim 95,
wherein the pharmaceutical composition is a drug or medicament.

97. A method for treating a human or an animal suffering from a disease of the human or animal body,
wherein the method comprises the administration of an efficient amount of at least one optionally C₅-C₃₄-fatty acid functionalized 3-hydroxybutyric acid polyglycerol ester of general formula (IIb)
R⁵O—CH₂—CH(OR⁵)—CH₂—[O—CH₂—CH(OR⁵)—CH₂]ₚ—OR⁵ (IIb)
wherein, in the general formula (IIb),
the variable p represents an integer from 1 to 4,
the radical R⁵, each independently of one another, identical or different, represents: hydrogen, a radical R², wherein the radical R² represents a radical of the type linear or branched, saturated or mono- or polyunsaturated (C₄-C₃₃-alkyl)-C(O)—, wherein the radical R⁶ represents hydrogen or a radical R² as defined hereinabove, however, with the proviso that at least one radical R⁵ does not represent hydrogen, and with the proviso that at least one radical R⁵ represents a radical CH₃— CH(OR⁶)—CH₂—C(O)—.

98. The method of claim 97,
wherein the disease is selected among diseases associated with a disorder of the energy metabolism or diseases associated with a disorder of the keto-body metabolism.

99. The method of claim 98,
wherein the disease is selected among craniocerebral trauma, stroke, hypoxia, cardiovascular diseases, myocardial infarction, refeeding syndrome, anorexia, epilepsy, neurodegenerative diseases, dementia, Alzheimer's disease, Parkinson's disease, multiple sclerosis and amyotrophic lateral sclerosis, fat metabolic diseases, glucose transporter defect (GLUT1 defect), VL-FAOD and mitochondriopathies, mitochondrial thiolase defect, Huntington's disease, cancers, T-cell lymphomas, astrocytomas and glioblastomas, HIV, rheumatic diseases, rheumatoid arthritis and arthritis urica, diseases of the gastrointestinal tract, chronic inflammatory bowel diseases, ulcerative colitis and Crohn's disease, lyosomal storage diseases, sphingolipidosis, Niemann-Pick disease, diabetes mellitus and effects or side-effects of chemotherapy.

100. A food or a food product comprising at least one optionally C₅-C₃₄-fatty acid functionalized 3-hydroxybutyric acid polyglycerol ester of general formula (IIb)
R⁵O—CH₂—CH(OR⁵)—CH₂—[O—CH₂—CH(OR⁵)—CH₂]ₚ—OR⁵ (IIb)
wherein, in the general formula (IIb),
the variable p represents an integer from 1 to 4,
the radical R⁵, each independently of one another, identical or different, represents: hydrogen, a radical R², wherein the radical R² represents a radical of the type linear or branched, saturated or mono- or polyunsaturated (C₄-C₃₃-alkyl)-C(O)—, wherein the radical R⁶ represents hydrogen or a radical R² as defined hereinabove, however, with the proviso that at least one radical R⁵ does not represent hydrogen, and with the proviso that at least one radical R⁵ represents a radical CH₃— CH(OR⁶)—CH₂—C(O)—.
